(19) United States
(12) Patent Application Publication
(10) Pub. No.: US 2025/0226108 A1
(43) Pub. Date: Jul. 10, 2025

(54) MULTI-TIERED TESTING FOR TRACKING CANCER HETEROGENEITY

(71) Applicant: Flagship Pioneering Innovations VI, LLC, Cambridge, MA (US)

(72) Inventor: Anthony P. Shuber, Cambridge, MA (US)

(21) Appl. No.: 19/009,567

(22) Filed: Jan. 3, 2025

Related U.S. Application Data
(60) Provisional application No. 63/636,405, filed on Apr. 19, 2024.
(60) Provisional application No. 63/617,989, filed on Jan. 5, 2024.

(51) Int. Cl. G16H 50/30; C12Q 1/6806; C12Q 1/6809; C12Q 1/686; C12Q 1/6886; G16B 20/20; G16B 30/10; G16B 40/10; G16B 40/20; G16H 20/10

(52) U.S. Cl. CPC G16H 50/30; C12Q 1/6806; C12Q 1/6809; C12Q 1/686; C12Q 1/6886; G16B 20/20; G16B 30/10; G16B 40/10; G16B 40/20; G16H 20/10; C12Q 2600/106; C12Q 2600/136; C12Q 2600/154

(57) ABSTRACT

Disclosed is a tiered, multipart method for tracking tumor heterogeneity across samples obtained from a subject at different timepoints. Each sample undergoes at least an intra-individual analysis to generate background-corrected methylation information. The change in the background-corrected methylation information across the different samples is informative for tracking a change in the tumor heterogeneity. The change in tumor heterogeneity is useful e.g., for providing a guided therapy.

### CROSS REFERENCE TO RELATED APPLICATIONS

This application claims the benefit of and priority to U.S. Provisional Patent Application No. 63/636,405 filed Apr. 19, 2024, and U.S. Provisional Patent Application No. 63/617,989 filed Jan. 5, 2024 the entire disclosure of each of which is hereby incorporated by reference in its entirety for all purposes.

### BACKGROUND

Diagnostic technologies include simple, point of care (POC) tests applied to large populations to identify relatively common diseases as well as complex, centralized tests applied to select populations. However, although POC tests can be applied to large populations, they are incapable of identifying individuals for cancer at a high enough accuracy to be feasible for implementation. Similarly, although complex, centralized testing can be deployed for rare population testing, such testing is often invasive, expensive, and fails when applied for detecting rare cancers in large patient populations. For example, complex, centralized testing suffers from poor performance (e.g., high number of false positives and/or low positive predictive value) when attempting to diagnose rare cancers in large patient populations. Thus, current POC tests are not suitable for identifying individuals with cancer and for tracking such individuals over time.

### SUMMARY

Disclosed herein are methods involving a multiple tiered analysis for tracking tumor heterogeneity in subjects. In particular, the methods disclosed herein involving a multiple tiered analysis are useful for tracking tumor heterogeneity in individuals from a large population (e.g., millions of individuals) who have a rare cancer. The multiple tiered analysis involves a first screen, which eliminates a large proportion of individuals who are identified as negative for cancer. For subjects that are identified as not negative for cancer, they can be provided an intervention (e.g., a tumor therapeutic). These subjects undergo additional analyses (e.g., one or more intra-individual analysis and/or a second analysis) which can be performed using samples obtained from the subjects across different timepoints. For example, intra-individual analyses can be conducted for each sample obtained from the subject. By doing so, a change in tumor heterogeneity can be determined which is informative for determining the efficacy of the provided intervention. Altogether, the multiple tiered analysis can be useful e.g., for guided therapy.

### BRIEF DESCRIPTION OF THE DRAWINGS

These and other features, aspects, and advantages of the present invention will become better understood with regard to the following description and accompanying drawings. It is noted that wherever practicable, similar or like reference numbers may be used in the figures and may indicate similar or like functionality. For example, a letter after a reference numeral, such as “third party entity 155A,” indicates that the text refers specifically to the element having that particular reference numeral. A reference numeral in the text without a following letter, such as “third party entity 155,” refers to any or all of the elements in the figures bearing that reference numeral (e.g. “third party entity 155” in the text refers to reference numerals “third party entity 155A” and/or “third party entity 155B” in the figures).

Figure (FIG.) 1A depicts an overall flow process of the multiple-tiered process for tracking tumor heterogeneity, in accordance with an embodiment.

FIG. 1B depicts an overall flow process of the multiple-tiered process for tracking tumor heterogeneity, in accordance with a second embodiment.

FIG. 1C depicts an overall system environment including a tumor heterogeneity system, in accordance with an embodiment.

FIG. 2A depicts a block diagram of the tumor heterogeneity system, in accordance with an embodiment.

FIG. 2B depicts an example conversion of nucleic acids, in accordance with an embodiment.

FIG. 2C shows the results of nitrite conversion on select nucleotides, in accordance with a second embodiment. Figure adapted from Li et al. (2022) Genome Biology 23:122.

FIG. 3A depicts example methylation information useful for determining whether an individual is at risk for cancer, in accordance with an embodiment.

FIG. 3B shows an example flow process for determining whether an individual is at risk for cancer, in accordance with an embodiment.

FIG. 3C depicts an example process of combining sequence information of target nucleic acids and reference nucleic acids to generate a signal informative for determining presence or absence of cancer, in accordance with an embodiment.

FIG. 3D is an illustrative example of a signal informative for cancer, in accordance with an embodiment.

FIG. 3E shows aligned sequence reads of an analyte and a corresponding window of a kmer size, in accordance with an embodiment.

FIG. 3F shows the generation of metrics from sequence reads across 2^{k} possible patterns, in accordance with an embodiment.

FIG. 3G shows an example data structure including information useful for training machine learning models, in accordance with an embodiment.

FIG. 4A shows an example flow process involving a first and second intra-individual analyses, in accordance with a first embodiment.

FIG. 4B shows an example flow process involving a first and second intra-individual analyses, in accordance with a second embodiment.

FIG. 5 illustrates an example computer for implementing the entities shown in FIGS. 1A-1C, 2A, 3A-3G, and 4A-4B.

FIG. 6 shows example performance of different tiers of the multiple tier analysis for diagnosing individuals with cancer (e.g., prostate cancer).

FIG. 7 depicts performance of a single tier analysis and a two-tier analysis of a population involving 1046 samples.

FIG. 8 shows an example sample from which target nucleic acids and reference nucleic acids are obtained.

### DETAILED DESCRIPTION

### Definitions

Terms used in the claims and specification are defined as set forth below unless otherwise specified.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “obtaining information,” “obtaining marker information,” and “obtaining sequence information” encompasses obtaining information that is determined from at least one sample. Obtaining information (e.g., marker information or sequence information) encompasses obtaining a sample and processing the sample to experimentally determine the information (e.g., marker information or sequence information). The phrase also encompasses receiving the information, e.g., from a third party that has processed the sample to experimentally determine the information.

The terms “marker,” “markers,” “biomarker,” and “biomarkers” encompass, without limitation, lipids, lipoproteins, proteins, cytokines, chemokines, growth factors, peptides, nucleic acids (e.g., DNA or RNA), genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a prediction model, or are useful in prediction models developed using related markers (e.g., non-mutated versions of the proteins or nucleic acids, alternative transcripts, etc.).

The term “screen” or a “first analysis” refers to a step in the first tier of a multiple tiered analysis. The screen achieves a high specificity and removes a large majority of true negatives (e.g., individuals not at risk of a cancer). In various embodiments, the “screen” refers to an in silico screen that involves application of a machine learning model. For example, such a machine learning model may analyze sequence information (e.g., methylation information) and predicts whether individuals are likely to be at risk of the cancer.

The phrase “second analysis” refers to a step in the second tier of a multiple tiered analysis. The second analysis is performed on individuals who were identified, using the screen, as not negative for cancer. Thus, the second analysis achieves a higher positive predictive value than the screen, given that the screen removes a large proportion of the true negatives. In various embodiments, the “second analysis” refers to an in silico analysis that involves application of a machine learning model that analyzes sequence information (e.g., methylation information). The second analysis can predict whether individuals have cancer. In various embodiments, the second analysis is implemented to predict a change in tumor heterogeneity for purposes of tracking tumor heterogeneity in a subject.

The phrase “intra-individual analysis” refers to an analysis performed for an individual that removes baseline biological signatures that are less informative for determining whether the individual is at risk for cancer. In various embodiments, the intra-individual analysis involves combining information from target nucleic acids and reference nucleic acids of an individual to generate a signal informative for determining presence or absence of cancer within the individual. By combining the information from the target nucleic acids and the reference nucleic acids, the generated signal can be more informative of presence or absence of cancer in comparison to a signal derived from the target nucleic acids alone.

The phrase “target nucleic acids” refers to nucleic acids of an individual that contain at least signatures that may be informative for determining presence or absence of cancer. The target nucleic acids may further include baseline biological signatures of the individual that are not informative or less informative. In various embodiments, target nucleic acids may be nucleic acids derived from a diseased cell that is associated with cancer. For example, target nucleic acids may be cell-free nucleic acids originating from cancer cells. Target nucleic acids can be any of DNA, cDNA, or RNA. In particular embodiments, target nucleic acids include DNA.

The phrase “reference nucleic acids” refers to nucleic acids of an individual that contain baseline biological signatures of the individual. Here, the baseline biological signatures of the individual may be present when the individual is healthy, and therefore, the baseline biological signatures are less informative for determining presence or absence of cancer in comparison to sequence information of the target nucleic acids. Reference nucleic acids can be any of DNA, cDNA, or RNA. In particular embodiments, reference nucleic acids include DNA.

It must be noted that, as used in the specification, the singular forms “a,” “an” and “the” include plural referents unless the context clearly dictates otherwise.

### Overview of Multiple Tier Analysis

Disclosed herein is a tiered, multipart method for tracking tumor heterogeneity across samples obtained from a subject at different timepoints. For example, methods disclosed herein are useful for detecting circulating tumor DNA from samples obtained from a subject across two or more timepoints. Determining the change in circulating tumor DNA from samples obtained from the subject across two or more timepoints enables tracking of the tumor heterogeneity. In various embodiments, tracking tumor heterogeneity is informative for determining whether an intervention (e.g., a tumor therapeutic) is efficacious. Therefore, tracking tumor heterogeneity can be useful for e.g., guided therapy.

In various embodiments, the tiered, multipart method involves performing a first analysis of nucleic acid sequence information that was derived from a first assay performed on a biological sample obtained from the subject. This first analysis identifies whether the biological sample is at risk or not at risk of containing circulating tumor DNA. In various embodiments, for a biological sample that is determined as not negative for containing circulating tumor DNA, the multipart method further includes performing an intra-individual analysis and a second analysis. In various embodiments, the intra-individual analysis includes obtaining target nucleic acids and reference nucleic acids from the biological sample or an additional biological sample obtained from the individual; processing the target nucleic acids and reference nucleic acids to generate a dataset comprising methylation information from the target nucleic acids and methylation information from the reference nucleic acids; and using a computer processor, combining the methylation information from the target nucleic acids and the methylation information from the reference nucleic acids to generate background-corrected methylation information for the target nucleic acids. Here, the background-corrected methylation information is more informative for determining presence or absence of cancer within the individual. In various embodiments, performing the second analysis comprises analyzing the background-corrected methylation information to detect the presence of the circulating tumor DNA in the biological sample. By detecting presence of circulating tumor DNA in the biological sample, the individual can be identified as having cancer.

Generally, multi-tier testing methodologies described herein achieve significant improvements in comparison to conventional testing methodologies (e.g., single tier testing methodologies). For example, the multi-tier testing methodologies described herein achieve improved performance metrics (e.g., sensitivity, specificity, positive predictive value (PPV), and/or negative predictive value (NPV)) in comparison to conventional methodologies. In particular embodiments, the combination of a first tier and a second tier testing achieves improved specificity (e.g., true negative rate reported as a proportion of correctly identified negatives) in comparison to conventional methodologies.

In some scenarios, the multi-tier testing methodologies described herein rapidly and accurately screen out a large proportion of individuals in a first tier through a more efficient, lower cost tier 1 test, followed by a more rigorous tier 2 test on the remaining subpopulation of patients. Here, the multi-tier testing methodology can achieve overall performance metrics that are comparable to or not substantially less than the overall performance metrics of conventional methodologies. Altogether, by rapidly and accurately screening out a large proportion of individuals in a first tier, only a small number of individuals undergo the more rigorous tier 2 testing. This represents an improvement in comparison to conventional methodologies that attempt to apply rigorous tests across the entire population, which requires substantial resources. Thus, even in scenarios where the multi-tier testing methodologies achieve performance metrics comparable to those of conventional methodologies, the multi-tier testing methodologies deliver improved performance as a function of resource consumption. Examples of resource consumption include time resources, monetary resources, resources of consumable goods (e.g., consumable assay reagents). In various embodiments, the multi-tier testing methodologies disclosed herein achieve at least a 10% reduction in resource consumption in comparison to a corresponding single-tier test. In various embodiments, the multi-tier testing methodologies disclosed herein achieve at least a 20% reduction, at least a 30% reduction, at least a 40% reduction, at least a 50% reduction, at least a 60% reduction, at least a 70% reduction, at least a 80% reduction, or at least a 90% reduction in resource consumption in comparison to a corresponding single-tier test. In various embodiments, the multi-tier testing methodologies disclosed herein achieve at least a 60% reduction in resource consumption in comparison to a corresponding single-tier test. In particular embodiments, the multiple-tiered process disclosed herein is useful for detecting rare or low incidence cancers. For example, the rare or low incidence cancers may have an incidence rate of 1 in 100, 1 in 1,000, 1 in 10,000 individuals, 1 in 100,000 individuals, 1 in 1,000,000 individuals, 1 in 10,000,000 individuals, 1 in 100,000,000 individuals or 1 in 1,000,000,000 individuals. Therefore, the disclosed multiple-tiered process represents a significant improvement over current methodologies that suffer from poor specificity or sensitivity which contributes to their inability to detect rare or low incidence conditions with sufficient positive predictive value.

In various embodiments, subjects that were not screened out in the first tier further undergo subsequent analysis to track tumor heterogeneity. For example, the intra-individual analysis may be performed again to analyze a second sample obtained from the same subject at a second timepoint. Here, the second timepoint is subsequent to a first timepoint when the first sample was obtained. Performing the intra-individual analysis using the second sample generates background-corrected methylation information for the second sample. Therefore, by comparing the background-corrected methylation information of the first sample to the background-corrected methylation information of the second sample, a change in the background-corrected methylation information across the two samples is generated. Here, the change in the background-corrected methylation information across the two samples is informative for the change in tumor heterogeneity across the two timepoints from when the two samples were respectively obtained.

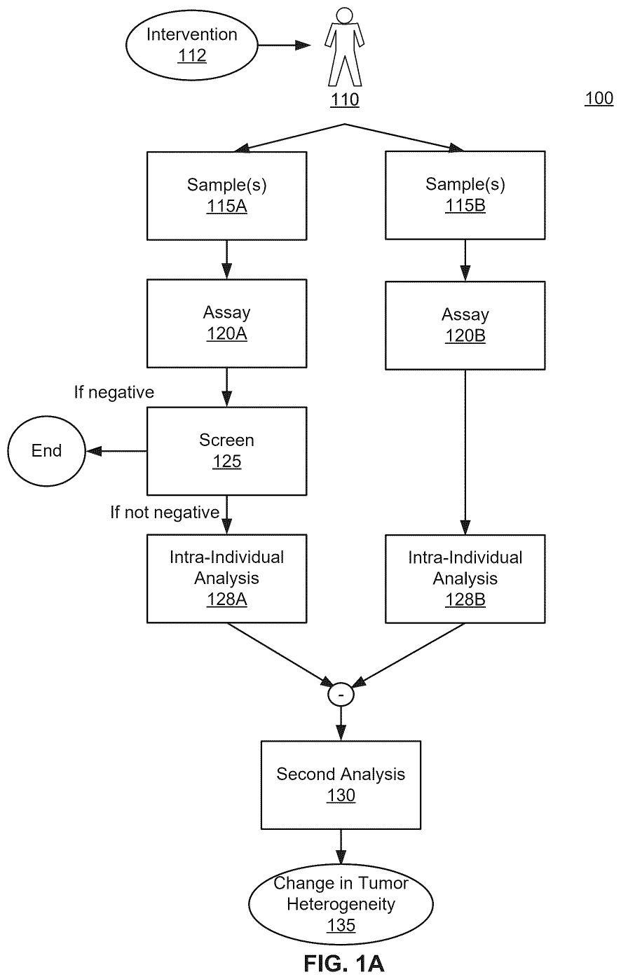

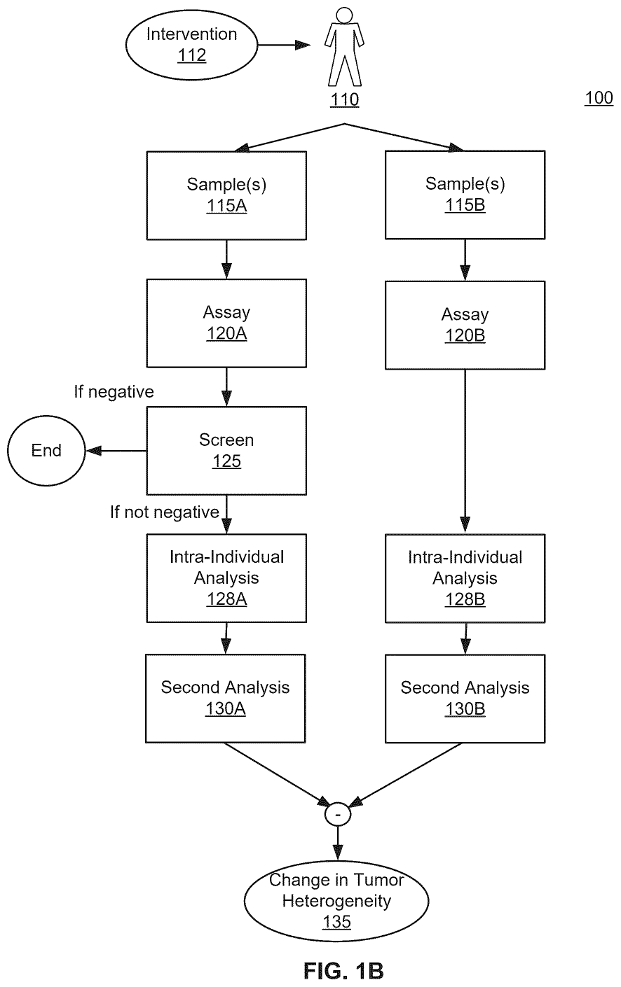

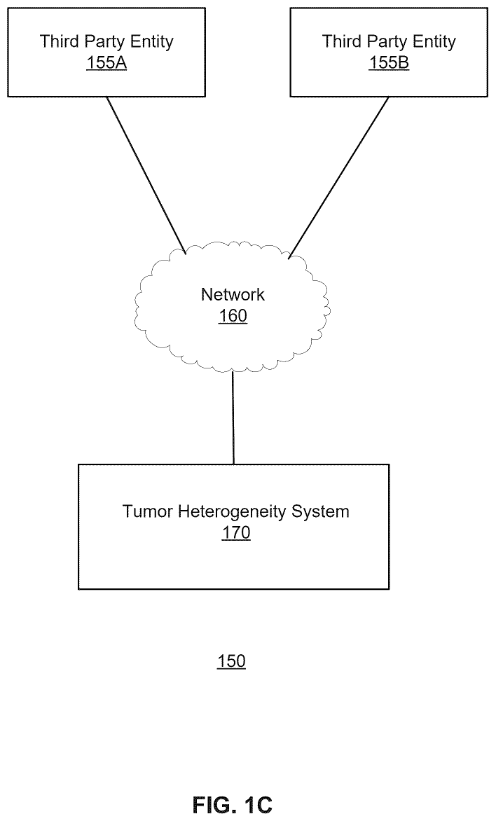

Figure (FIG.) 1A depicts an overall flow process 100 of the multiple-tiered process for tracking tumor heterogeneity, in accordance with an embodiment. Although FIG. 1A shows the flow process in relation to a single subject 110, in various embodiments, the flow process can be performed for more than a single subject 110 (e.g., for thousands, millions, tens of millions, or hundreds of millions of individuals).

FIG. 1A introduces a first sample 115A, an assay 120A, a first tier (e.g., screen 125), an intra-individual analysis 128A, a second sample 115B, an assay 120B, and a second tier (e.g., second analysis 130) of the multiple-tiered analysis. Generally, the second tier involves a more complex molecular test and analysis in comparison to the first tier. In various embodiments, the more complex molecular test of the second tier is more expensive to perform than the simpler molecular test of the first tier. By employing a cheaper and less complex test, the first tier can identify and remove of individuals that are not at risk of cancer. The more complex molecular test and analysis of the second tier enables more accurate identification of the remaining individuals for purposes of tracking tumor heterogeneity. As shown in FIG. 1A, the method may involve two or more intra-individual analyses performed on different samples. Here, an intra-individual analysis removes baseline biological signatures. For example, the intra-individual analysis can be performed to remove baseline biological signatures in sequencing information (hereafter referred to as “background-corrected information”) prior to the performance of the second tier. Thus, the more complex molecular test of the second tier can be applied to analyze the background-corrected information of two or more intra-individual analyses to more accurately track tumor heterogeneity in a subject.

Although FIG. 1A shows a first tier and a second tier of a multiple-tiered analysis, in various embodiments, there may be additional tiers for further classifying individuals. In various embodiments, the multiple-tiered analysis includes three or more tiers, includes four or more tiers, includes five or more tiers, includes six or more tiers, includes seven or more tiers, includes eight or more tiers, includes nine or more tiers, or includes ten or more tiers.

In various embodiments, the combination of the first tier and the second tier enables the ultimate high performance (e.g., high positive predictive value) of the multiple-tier analysis. In various embodiments, the first tier and the second tier interrogate different markers from samples obtained from subjects. This can be beneficial because different markers can provide different information. In some cases, different markers can be informative for different predictions. As an example, the first tier may analyze protein markers from samples obtained from subjects whereas the second tier may analyze sequencing data derived from nucleic acids in the samples obtained from subjects.

In various embodiments, the first tier and second tier interrogate the same type of markers from samples obtained from subjects, but at different levels of detail. For example, the first tier may involve the analysis of methylation statuses for a limited, pre-selected set of genomic sites. The differential methylation of the limited, pre-selected set of genomic sites is sufficient to enable identification of subjects not at risk of cancer. Additionally, the second tier may involve the analysis of methylation statuses for a larger set of genomic sites. In one scenario, the second tier involves analysis of methylation statuses for the whole genome (e.g., through whole genome bisulfite sequencing). The differential methylation of the larger set of genomic sites enables more accurate tracking of tumor heterogeneity in the remaining subjects. As another example, the first tier may involve the analysis of shallow sequencing data. Here, shallow sequencing data is sufficient to identify and remove subjects who are not at risk or who do not have cancer. The second tier may involve analysis of sequencing data derived from deeper sequencing, which is sufficient to track tumor heterogeneity for subjects who have cancer.

FIG. 1A introduces a subject 110. One or more samples (e.g., sample 115A and/or sample 115B) are obtained from the subject 110. In various embodiments, a sample is any of a blood sample, a stool sample, a urine sample, a mucous sample, or a saliva sample. In particular embodiments, each sample obtained from the subject 110 is a blood sample. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the one or more samples can be obtained from the subject 110 by a reference lab.

In various embodiments, the sample obtained from the subject is a liquid biopsy sample obtained at a first point in time. In various embodiments, the liquid biopsy sample may include various biomarkers, examples of which include proteins, metabolites, and/or nucleic acids. In particular embodiments, the liquid biopsy sample includes cell-free DNA (cfDNA) fragments. In particular embodiments, the cfDNA fragments include genomic sequences corresponding to CpG islands for which methylation states are informative of the cancer.

In various embodiments, a plurality of samples are obtained from the subject 110 at a plurality of different points in time. For example, a sample (e.g., sample 115A) can be obtained at a first timepoint and at least a second sample (e.g., sample 115B) can be obtained from the subject 110 at a second timepoint. In such embodiments, the first sample can be used for performing the assay 120A, the screen 125, and the intra-individual analysis 128A. Additionally, the second sample 115B can be used to perform an assay 120B, and a second intra-individual analysis 128B. The second analysis 130 can then be performed using the results from each of the two or more intra-individual analyses (e.g., intra-individual analysis 128A and intra-individual analysis 128B). Obtaining a plurality of liquid biopsy samples from the individual at a plurality of different points in time includes obtaining a number M of liquid biopsy samples, wherein M is one of: 2, 3, 4, . . . , N-1, N, wherein N is a positive integer.

In various embodiments, sample 115A and/or sample 115B may be processed to extract target nucleic acids and reference nucleic acids. In various embodiments, samples can undergo cellular disruption methods (e.g., to obtain genomic DNA) involving chemical methods or mechanical methods. Example chemical methods include osmotic shock, enzymatic digestion, detergents, or alkali treatment. Example mechanical methods include homogenization, ultrasonication or cavitation, pressure cell, or ball mill. In various embodiments, samples can undergo removal of membrane lipids or proteins or nucleic acid purification. Example chemical methods for removing membrane lipids or proteins and methods for nucleic acid purification include guanidine thiocyanate (GuSCN)-phenol-chloroform extraction, alkaline extraction, cesium chloride gradient centrifugation with ethidium bromide, Chelex® extraction, or cetyltrimethylammonium bromide extraction. Example physical methods for removing membrane lipids or proteins and methods for nucleic acid purification include solid-phase extraction methods using any of silica matrices, glass particles, diatomaceous earth, magnetic beads, anion exchange material, or cellulose matrix. Further details of nucleic acid extraction methods are described in Ali et al, Current Nucleic Acid Extraction Methods and Their Implications to Point-of-Care Diagnostics, Biomed Res. Int. 2017; 2017:9306564, which is hereby incorporated by reference in its entirety.

Assay 120A and/or assay 120B are performed on the obtained sample 115A and 115B, respectively, to generate marker information. An example of marker information can include quantitative levels of a biomarker, such as a protein biomarker, nucleic acid biomarker, metabolite biomarker, that is present in the sample. Another examples of marker information is sequence information for a plurality of genomic sites. In various embodiments, given that the assay 120 may be performed on a large number of samples (e.g., millions of samples) obtained from a large patient population, the assay 120 be a simplified molecular test that generates marker information that can rapidly distinguish between individuals at risk and individuals not at risk for cancer. For example, the marker information can include quantitative levels of a biomarker, such as a protein biomarker, nucleic acid biomarker, metabolite biomarker, that can rapidly guide the identification and removal of individuals not at risk for the cancer. As another example, the marker information can be sequence information for a limited number of genomic sites that are sufficient for identifying individuals who are not at risk for the cancer (e.g., true negatives). In particular embodiments, the sequence information for a plurality of genomic sites includes methylation information, such as methylation statuses for the plurality of genomic sites. In various embodiments, the plurality of genomic sites include a plurality of CpG islands (CGIs) whose differential methylation status may be indicative of risk for the cancer.

In particular embodiments, assay 120A and/or assay 120B are performed to generate sequence information for target nucleic acids and to generate sequence information for reference nucleic acids. Thus, sequence information of target and reference nucleic acids can be used to perform the intra-individual analysis 128A and/or intra-individual analysis 128B. In particular embodiments, sequence information includes statuses for a plurality of genomic sites, such as epigenetic statuses for a plurality of CpG sites. In various embodiments, epigenetic statuses refer to methylation statuses. In particular embodiments, sequence information of the target nucleic acids and sequence information of the reference nucleic includes statuses for two or more, three or more, four or more, five or more, six or more, seven or more, eight or more, nine or more, or ten or more common genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more of the same genomic sites or overlapping genomic sites. In various embodiments, the plurality of genomic sites include a plurality of CpG islands (CGIs) whose differential methylation status may be indicative of a cancer.

A screen 125 is performed to analyze the marker information generated by the assay 120A. For example, the screen 125 can involve an in silico analysis of the marker information. In various embodiments, the marker information includes quantitative values of biomarkers. Therefore, the screen 125 can identify and remove individuals whose quantitative values of biomarkers indicate that the individuals are not at risk of the cancer. In various embodiments, the marker information is sequence information for a plurality of genomic sites. Therefore, the screen 125 involves deploying a trained machine learning model that analyzes the sequence information for the plurality of genomic sites and predicts whether an individual is at risk for a cancer. If the screen 125 identifies the individual as not at risk for cancer (as indicated in FIG. 1A as “If negative”), then the subject 110 can be reported as not at risk for the cancer. The process can terminate for this subject and therefore, additional resources need not be further devoted to this subject.

Alternatively, if the screen identifies the subject as at risk for cancer (as indicated in FIG. 1A as “If not negative” following screen 125), then the subject 110 undergoes at least another tier of testing. As shown in FIG. 1A, an intra-individual analysis 128A and a second analysis 130 can be performed for subjects identified as at risk for cancer. In particular embodiments, a second sample 115B, assay 120B and second intra-individual analysis 128B are performed for the subject after having determined that the subject is not negative based on the results of the screen 125.

In various embodiments, as shown in FIG. 1A, the subject 110 receives an intervention 112. In various embodiments, the subject 110 receives the intervention 112 after the screen determines that the subject 110 is not negative for cancer. Thus, the subject 110 may have been selected and provided the intervention to treat for the cancer and/or to reduce the risk for cancer. An example of an intervention 112 is a tumor therapeutic (e.g., a cancer therapeutic, a chemotherapy, and/or a gene therapy).

Referring to the intra-individual analysis 128A and intra-individual analysis 128B, the analysis is conducted for a specific subject, such as a subject identified via the screen 125 as at risk for the cancer. Therefore, for a particular subject, the intra-individual analysis is performed to remove baseline biological signatures that are present in the subject. Here, the baseline biological signatures are present irrespective of whether the subject has or does not have cancer. These baseline biological signatures would be confounding signals if analyzed to generate predictions for the patient. Thus, performing the intra-individual analysis 128 for individual samples (e.g., sample 115A or sample 115B) eliminates these confounding baseline biological signatures while keeping signatures that are more informative for determining presence or absence of cancer. For example, in processing nucleic acid sequencing information to generate a signal that may be detected, the resulting signal may comprise a mixture of baseline biological signatures (e.g., germline methylation in a patient) that represent a form of background noise and signatures informative of a cancer(e.g., cancer). Such background noise can obscure a signal informative of a cancer. Advantageously, in certain embodiments, methods described herein contemplate subtracting such background noise from a patient's nucleic acid sequencing information, thereby improving the signal-to-noise ratio of the signal informative of a cancer.

In contrast to an inter-individual analysis, where, for example, to determine a presence or absence of cancer within a patient, an average of baseline signatures from a group of normal subjects are removed from the nucleic acid sequencing information of the patient, it has been discovered that performing an intra-individual analysis can significantly improve the sensitivity or specificity of detecting a signal informative for determining presence or absence of cancer.

Generally, the intra-individual analysis 128A or intra-individual analysis 128B involves generating information from at least target nucleic acids and reference nucleic acids from a corresponding sample (e.g., sample 115A and sample 115B) obtained from the patient. In various embodiments, the intra-individual analysis 128A and intra-individual analysis 128B is performed on sequence information. Such sequence information may be generated by assay 120A and assay 120B, as shown in FIG. 1A.

In various embodiments, the intra-individual analysis 128A and intra-individual analysis 128B involve combining information from target nucleic acids and the reference nucleic acids to generate a signal informative for determining presence or absence of cancer within the patient. By combining the information from the target nucleic acids and the reference nucleic acids, the generated signal can be more informative of presence or absence of a cancer in comparison to a signal derived from the target nucleic acids alone. For example, the information from the reference nucleic acids can represent baseline biology of the patient. By combining the information from the target nucleic acids and the reference nucleic acids, the baseline biology of the patient, which may not be informative for the presence or absence of a cancer, is removed from the generated signal. Thus, information of the target nucleic acids that are not attributable to the patient's baseline biology remains and is included in the generated signal for determining presence or absence of cancer in the patient.

Referring next to the second analysis 130, the second analysis 130 is implemented to determine a change in tumor heterogeneity 135 in the subject 110. In various embodiments, the second analysis 130 determines a change in signal between a first set of background-corrected methylation information generated from the first intra-individual analysis 128A and a second set of background-corrected methylation information generated from the second intra-individual analysis 128B. For example, as shown in FIG. 1A, the output of each of the intra-individual analysis 128A and intra-individual analysis 128B can be combined to determine the change in signal. The change in signal can be provided for the second analysis 130 and can be indicative of whether the tumor heterogeneity in the subject is increasing, decreasing, or remaining stable.

Referring next to FIG. 1B, it depicts an overall flow process of the multiple-tiered process for tracking tumor heterogeneity, in accordance with a second embodiment. Here, FIG. 1B differs from FIG. 1A in that the second analysis 130 is individually performed to analyze the results of each respective intra-individual analysis e.g., intra-individual analysis 128A and intra-individual analysis 128B. Therefore, as shown in FIG. 1B, the output of the second analysis 130A can be combined with the output of second analysis 130B to determine a change in tumor heterogeneity 135 for the subject 110.

Altogether, the multiple-tiered analysis (e.g., multiple-tiered analysis involving the screen 125 and second analysis 130 or multiple-tiered analysis involving each of the screen 125, intra-individual analysis 128, and second analysis 130) enables the rapid identification of a large proportion of individuals (e.g., greater than 80% of the patient population) representing true negatives, and further enables the accurate identification and diagnosis of a subset of the population representing true positives. The overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves one or more performance metrics, such as metrics of sensitivity, specificity, positive predictive value (PPV), and/or negative predictive value (NPV). Sensitivity is the true positive rate, reported as a proportion of correctly identified positives. Specificity is the true negative rate reported as a proportion of correctly identified negatives. Positive predictive value refers to the number of true positives divided by the sum of true positives and false positives. Negative predictive value refers to the true negative rate divided by the sum of true negatives and false negatives.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves at least 60% sensitivity in detecting presence of a cancer. In various embodiments, the overall multiple-tiered analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the overall multiple-tiered analysis achieves at least 70% sensitivity. In particular embodiments, the overall multiple-tiered analysis achieves at least 71% sensitivity. In particular embodiments, the overall multiple-tiered analysis achieves at least 72% sensitivity. In particular embodiments, the overall multiple-tiered analysis achieves at least 73% sensitivity. In particular embodiments, the overall multiple-tiered analysis achieves at least 74% sensitivity. In particular embodiments, the overall multiple-tiered analysis achieves at least 75% sensitivity.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves at least 60% specificity in excluding individuals without the cancer. In various embodiments, the overall multiple-tiered analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the overall multiple-tiered analysis achieves at least 99% specificity. In particular embodiments, the overall multiple-tiered analysis achieves at least 99.5% specificity. In particular embodiments, the overall multiple-tiered analysis achieves at least 99.9% specificity.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves a particular sensitivity and a particular specificity. The combination of the sensitivity and specificity limits both the number of false positives and the number of false negatives. In various embodiments, the overall multiple-tiered analysis achieves between 70% to 90% sensitivity and between 90% to 100% specificity. In various embodiments, the overall multiple-tiered analysis achieves between 75% to 89% sensitivity and between 90% to 100% specificity. In various embodiments, the overall multiple-tiered analysis achieves between 80% to 88% sensitivity and between 90% to 100% specificity. In various embodiments, the overall multiple-tiered analysis achieves between 83% to 87% sensitivity and between 90% to 100% specificity. In various embodiments, the overall multiple-tiered analysis achieves between 84% to 86% sensitivity and between 90% to 100% specificity. In various embodiments, the overall multiple-tiered analysis achieves about 85% sensitivity and between 90% to 100% specificity.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves between 70% to 90% sensitivity and between 91% to 99% specificity. In various embodiments, the overall multiple-tiered analysis achieves between 70% to 90% sensitivity and between 92% to 98% specificity. In various embodiments, the overall multiple-tiered analysis achieves between 70% to 90% sensitivity and between 93% to 97% specificity. In various embodiments, the overall multiple-tiered analysis achieves between 70% to 90% sensitivity and between 97% to 96% specificity. In various embodiments, the overall multiple-tiered analysis achieves between 70% to 90% sensitivity and about 95% specificity.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves between 75% to 89% sensitivity and between 91% to 99% specificity. In various embodiments, the overall multiple-tiered analysis achieves between 80% to 88% sensitivity and between 92% to 98% specificity. In various embodiments, the overall multiple-tiered analysis achieves between 83% to 87% sensitivity and between 93% to 97% specificity. In various embodiments, the overall multiple-tiered analysis achieves between 84% to 86% sensitivity and between 94% to 96% specificity. In various embodiments, the overall multiple-tiered analysis achieves about 85% sensitivity and about 95% specificity.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves at least 60% positive predictive value. In various embodiments, the overall multiple-tiered analysis achieves at least 20% positive predictive value. In various embodiments, the overall multiple-tiered analysis achieves at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% positive predictive value. In various embodiments, the overall multiple-tiered analysis achieves at least 40% positive predictive value. In various embodiments, the overall multiple-tiered analysis achieves at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, or at least 60% positive predictive value. In various embodiments, the overall multiple-tiered analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In particular embodiments, the overall multiple-tiered analysis achieves at least 80% positive predictive value. In particular embodiments, the overall multiple-tiered analysis achieves at least 81% positive predictive value. In particular embodiments, the overall multiple-tiered analysis achieves at least 82% positive predictive value. In particular embodiments, the overall multiple-tiered analysis achieves at least 83% positive predictive value. In particular embodiments, the overall multiple-tiered analysis achieves at least 84% positive predictive value. In particular embodiments, the overall multiple-tiered analysis achieves at least 85% positive predictive value.

In various embodiments, the overall multiple-tiered analysis (e.g., multiple-tiered analysis involving the screen 125 and second analysis 130 or multiple-tiered analysis involving each of the screen 125, intra-individual analysis 128A, intra-individual analysis 128B, and second analysis 130) achieves at least 60% negative predictive value. In various embodiments, the overall multiple-tiered analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the overall multiple-tiered analysis achieves at least 98% negative predictive value. In particular embodiments, the overall multiple-tiered analysis achieves at least 99% negative predictive value. In particular embodiments, the overall multiple-tiered analysis achieves at least 99.4% negative predictive value.

### System Environment Overview

FIG. 1C depicts an overall system environment 150 including a tumor heterogeneity system 170, in accordance with an embodiment. The overall system environment 150 includes a tumor heterogeneity system 170 for at least performing one or more steps shown in FIG. 1A, and one or more third party entities 155A and 155B in communication with one another through a network 160. FIG. 1B depicts one embodiment of the overall system environment 150 in which two third party entities 155A and 155B are involved. In other embodiments, additional or fewer third party entities 155 in communication with the tumor heterogeneity system 170 can be included. The third party entities 155 may communicate with the tumor heterogeneity system 170 to enable the tumor heterogeneity system 170 to perform a screen, one or more intra-individual analyses, and/or second analysis.

### Third Party Entity

A third party entity 155 represents a partner entity of the tumor heterogeneity system 170 that can operate upstream, downstream, or both upstream and downstream of the operations of the tumor heterogeneity system 170. As one example, the third party entity 155 operates upstream of the tumor heterogeneity system 170 and provides samples obtained from patients to the tumor heterogeneity system 170. Thus, the tumor heterogeneity system 170 can perform assays, a screen, one or more intra-individual analyses, and/or a second analysis to track tumor heterogeneity of subjects. As another example, the third party entity 155 may process samples obtained from subjects by performing one or more assays on the samples to generate data. Thus, the third party entity 155 can provide the data derived from the assays to the tumor heterogeneity system 170 such that the tumor heterogeneity system 170 can perform a screen, one or more intra-individual analyses, and/or second analysis.

As another example, the third party entity 155 operates downstream of the tumor heterogeneity system 170. In this scenario, the tumor heterogeneity system 170 may perform a screen and determine whether a subject is at risk for cancer. The tumor heterogeneity system 170 can provide an indication to the third party entity 155 that identifies the subject at risk for the cancer. The third party entity 155 may notify the subject regarding a follow-up appointment such that an additional sample (e.g., sample 115B shown in FIG. 1A) can be obtained from the subject at the follow-up appointment for subsequent analysis.

### Network

This disclosure contemplates any suitable network 160 that enables connection between the tumor heterogeneity system 170 and third party entities 155. The network 160 may comprise any combination of local area and/or wide area networks, using both wired and/or wireless communication systems. In one embodiment, the network 160 uses standard communications technologies and/or protocols. For example, the network 160 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 160 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 160 may be represented using any suitable format, such as hypertext markup language (HTML) or extensible markup language (XML). In some embodiments, all or some of the communication links of the network 160 may be encrypted using any suitable technique or techniques.

### Tumor Heterogeneity System

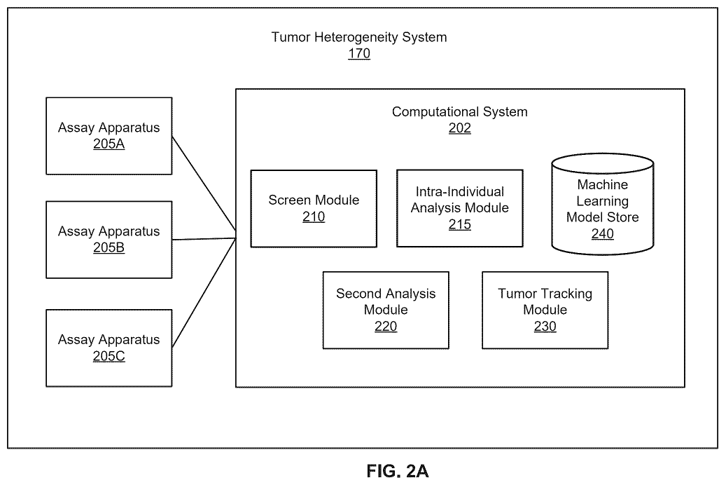

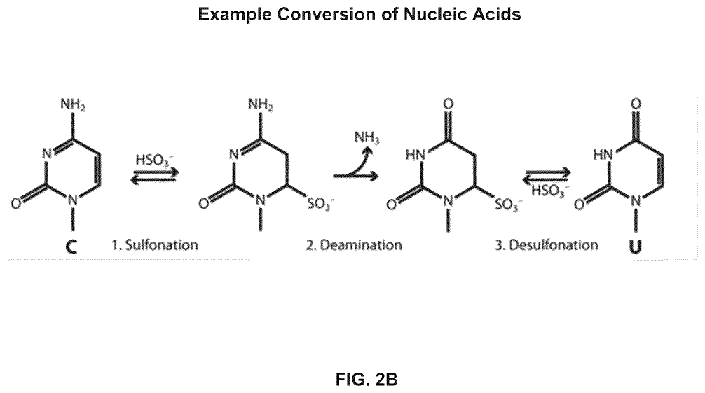

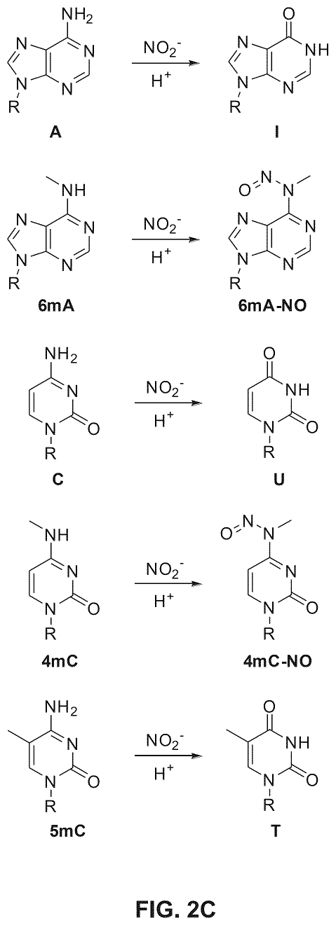

FIG. 2A depicts a block diagram of the tumor heterogeneity system 170, in accordance with an embodiment. The block diagram of the tumor heterogeneity system 170 is introduced to show an embodiment in which the tumor heterogeneity system 170 includes one or more assay apparatuses 205 communicatively coupled to a computational system 202. The computational system 202 can further include computational modules, such as a screen module 210, intra-individual analysis module 215, second analysis module 220, and a tumor tracking module 230. The computational system 202 can further include data stores such as a machine learning model store 240 for storing one or more trained machine learning models. FIG. 2A depicts an embodiment in which the tumor heterogeneity system 170 performs one or more assays (e.g., assay 120A or 120B described in FIG. 1A), performs the screen (e.g., screen 125 described in FIG. 1A), performs the one or more intra-individual analyses (e.g., intra-individual analysis 128A and/or intra-individual analysis 128B described in FIG. 1A), and performs the second analysis (e.g., second analysis 130 described in FIG. 1A).

In various embodiments, the tumor heterogeneity system 170 may be differently configured than shown in FIG. 2A. For example, although the tumor heterogeneity system 170 shown in FIG. 2A includes three different assay apparatuses 205, in various embodiments, the tumor heterogeneity system 170 includes fewer or additional assay apparatuses. In various embodiments, the tumor heterogeneity system 170 does not include an assay apparatus. In such embodiments, the tumor heterogeneity system 170 includes only the computational system 202. In these embodiments in which the tumor heterogeneity system 170 does not include an assay apparatus, the tumor heterogeneity system 170 may perform the screen (e.g., screen 125 described in FIG. 1A), one or more intra-individual analyses (e.g., intra-individual analysis 128A and/or intra-individual analysis 128B described in FIG. 1A), and the second analysis (e.g., second analysis 130 described in FIG. 1A). However, the tumor heterogeneity system 170 does not perform an assay. The assay apparatus 205 may be operated and used by a different entity, such as a third party entity (e.g., third party entity 155 described in FIG. 1C). Thus, the third party entity can perform assays using one or more assay apparatus 205 and then transmits the data generated from the assays to the tumor heterogeneity system 170 for performing the screen and/or second analysis.

### Assays

Methods disclosed herein involve performing an assay to generate marker information. Assays described in this section can refer to either assay 120A, assay 120B, or both assay 120A and assay 120B shown in FIGS. 1A and 1B. Referring to FIG. 2A, performing an assay can involve employing one or more assay apparatuses 205 to perform the assay. In various embodiments, marker information refers to quantitative values of biomarkers, such as protein biomarkers, nucleic acid biomarkers, or metabolite biomarkers. Thus, the quantitative values of biomarkers in a sample can be used to determine whether the individual is at risk for a cancer. In various embodiments, to determine quantitative values of protein biomarkers, performing an assay can include performing one or more of an immunoassay, a protein-binding assay, an antibody-based assay, an antigen-binding protein-based assay, a protein-based array, an enzyme-linked immunosorbent assay (ELISA), or a Western blot. To determine quantitative values of nucleic acid biomarkers, performing an assay can include performing one or more of quantitative PCR (qPCR) or digital PCR (dPCR). To determine quantitative values of metabolites, performing an assay can include performing NMR, mass spectrometry, LC-MS, or UPLC-MS/MS.

In various embodiments, marker information refers to sequence information for a plurality of genomic sites. The sequence information can then be analyzed to generate a prediction for an individual (e.g., whether an individual is negative for cancer or whether the individual is not negative for cancer). In particular embodiments, performing the assay results in generation of methylation sequence information. Methylation sequence information includes methylation statuses for a plurality of genomic sites. In various embodiments, the plurality of genomic sites are previously identified and selected. For example, the plurality of genomic sites may be one or more CpG sites whose differential methylation are informative for determining whether an individual is at risk for a cancer. A CpG site is portion of a genome that has cytosine and guanine separated by only one phosphate group and is often denoted as “5′-C-phosphate-G-3″”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.”

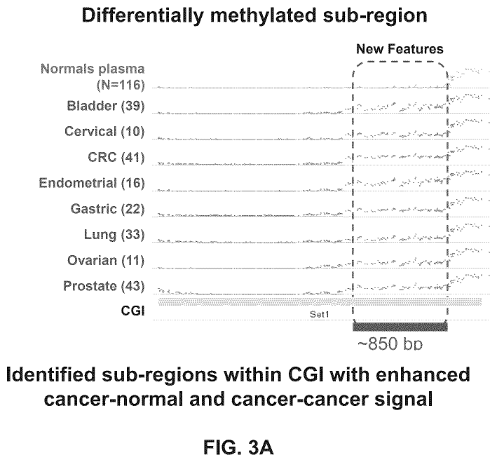

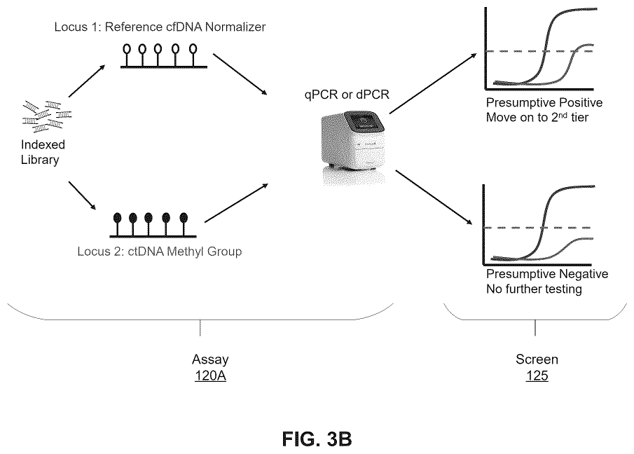

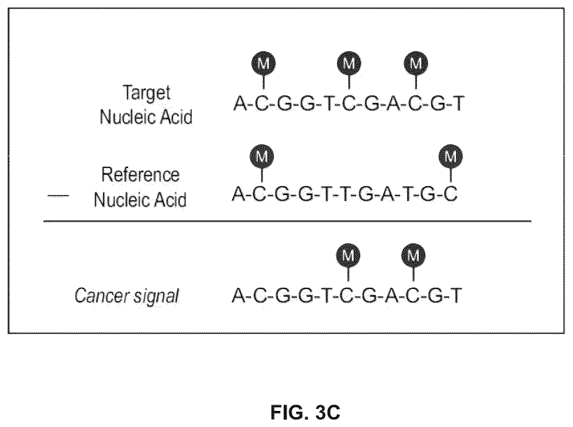

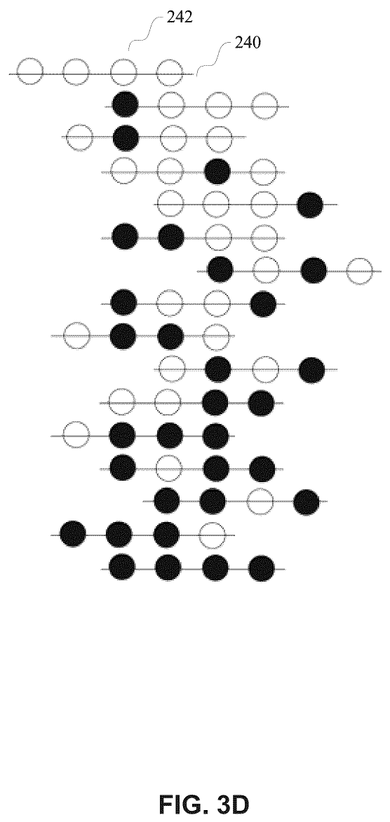

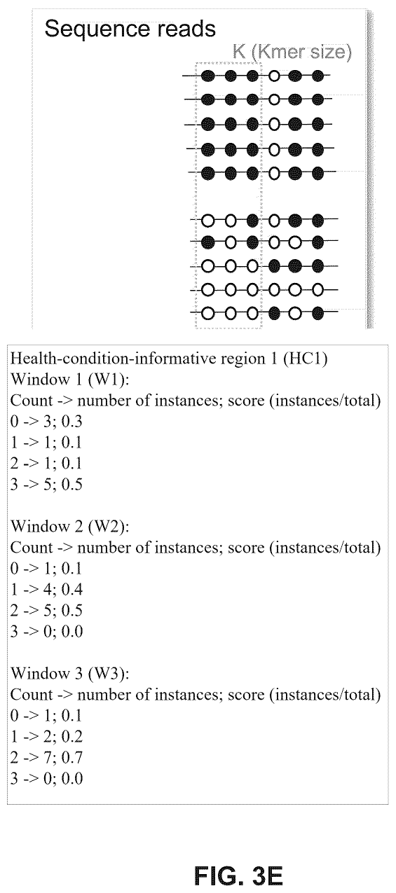

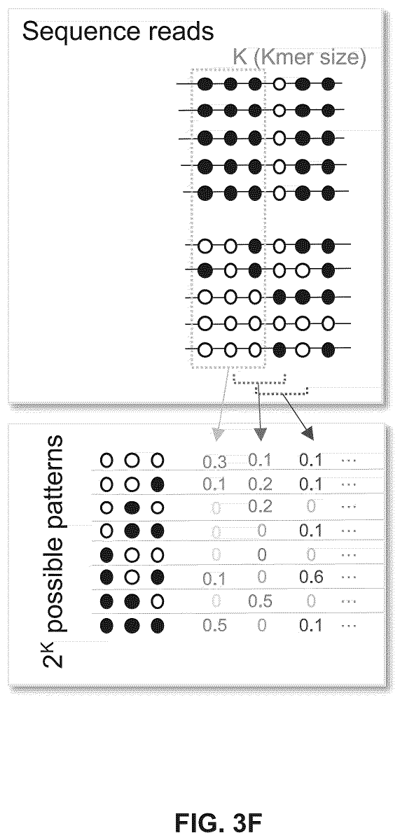

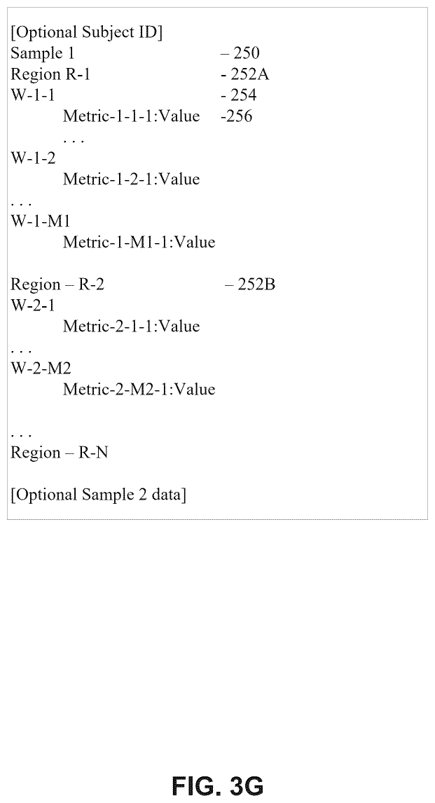

Reference is made to FIG. 3A, which depicts example methylation information useful for determining whether an individual is at risk for a cancer, in accordance with an embodiment. Specifically, FIG. 3A shows that across various types of cancers (e.g., bladder, cervical, colorectal, endometrial, gastric, lung, ovarian, and prostate cancers), sub-regions within a particular CGI can exhibit differential methylation in comparison to normal plasma. Thus, FIG. 3A depicts an example cancer informative CGI such that performing the assay results in the generation of methylation sequence information corresponding to the cancer informative CGI.

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing or via quantitative methods such as an ELISA, quantitative PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

Referring again to FIG. 1A or 1B, in various embodiments, assay 120A and assay 120B may both involve performing steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences. In various embodiments, assay 120A and assay 120B involve quantifying the amplicons by performing an ELISA assay, by performing quantitative PCR, or by performing next generation sequencing.

A methylated nucleic acid is a nucleic acid having a modification in which a hydrogen atom on the pyrimidine ring of a cytosine base is converted to a methyl group, forming 5-methylcytosine. Methylation can occur at dinucleotides of cytosine and guanine referred to herein as “CpG sites”, which can be a target for enrichment. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N⁶-methyladenine (6 mA). Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

In certain embodiments, the nucleic acid comprises a CpG site (i.e., cytosine and guanine separated by only one phosphate group). In certain embodiments, the nucleic acid comprises a CpG island (also referred to as a “CG islands” or “CGI”) or a portion thereof, which is the target for enrichment. Because certain CGIs and certain features of certain CGIs in tumor cells tend to be different from the same CGIs or features of the CGIs in healthy cells, detection of such CGIs can be informative of a cancer. In certain embodiments, the CGI is a “cancer informative CGIs”, which is defined and described in more detail below. In certain embodiments, the CpG is an “informative CpG”, e.g., a “cancer informative CGI”. Such CGIs may have methylation patterns in tumor cells that are different from the methylation patterns in healthy cells. Accordingly, detection of a cancer informative CGI can be informative regarding a subject's risk of developing cancer or can be indicative that the subject has cancer. Exemplary cancer informative CGIs, which can be target sequences as described herein, are identified in, e.g., Table 1 of U.S. Patent Publication 2020/0109456A1, Tables 2 and 3 of WO2022/133315, and Tables 1-4 provided herein.

In certain aspects, the nucleic acids have been treated to convert one or more unmethylated nucleotides (e.g., cytosines) to another nucleotide (a “converted nucleotide”, as used herein, such as a uracil), for example, prior to amplification. Example conversions include bisulfite conversion, enzymatic conversion, or nitrite conversion, further details of which are described herein. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5 mC)) is converted to a thymine, which pairs with adenine. In certain embodiments, methylated cytosines are protected from conversion (e.g., deamination) during the conversion step.

In various embodiments, nucleic acids undergo a bisulfite conversion. Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2B, the steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. (See Clark et al., Nucleic Acids Res., 22(15):2990-7 (1994).) If the cytosine is methylated it will remain a methylated cytosine. If the cytosine is unmethylated it will be converted to uracil. When the modified strand is copied, for example, through extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

In various embodiments, nucleic acids undergo an enzymatic conversion. In certain embodiments, the enzymatic treatment with a cytidine deaminase enzyme is used to convert cytosine to uracil. Enzymatic conversion can include an oxidation step, in which Tet methylcytosine dioxygenase 2 (TET2) catalyzes the oxidation of 5 mC to 5 hmC to protect methylated cytosines from conversion by subsequent exposure to a cytidine deaminase. Other protection steps known in the art can be used in addition to or in place of oxidation by TET2. After the oxidation step, the nucleic acid is treated with the cytidine deaminase to convert one or more unmethylated cytosines to uracils. As with bisulfite conversion, when the modified strand is copied, a G will be incorporated in the interrogation position (opposite the C being interrogated) if the C was methylated and an A will be incorporated in the interrogation position if the C was unmethylated. When the double stranded extension product is amplified those Cs that were converted to Us and resulted in incorporation of A in the extended primer will be replaced by Ts during amplification. Those Cs that were not modified and resulted in the incorporation of G will remain as C.

In certain embodiments the cytidine deaminase may be APOBEC. In certain embodiments the cytidine deaminase includes activation induced cytidine deaminase (AID) and apolipoprotein B mRNA editing enzymes, catalytic polypeptide-like (APOBEC). In certain embodiments, the APOBEC enzyme is selected from the human APOBEC family consisting of: APOBEC-1 (Apo1), APOBEC-2 (Apo2), AID, APOBEC-3A, -3B, -3C, -3DE,-3F, -3G, -3H and APOBEC-4 (Apo4). In certain embodiments, the APOBEC enzyme is APOBEC-seq.

In certain embodiments, nitrite treatment is used to deaminate adenine and cytosine. As shown in FIG. 2C, deamination of an A results in conversion to an inosine (I), which is read by a polymerase as a G, whereas deamination of a methylated A (N-methyladenine (6 mA)) results in a nitrosylated 6 mA (6 mA-NO), which causes the base to be read by a polymerase as an A. Deamination of a C results in conversion to a uracil, which is read by a polymerase as a T, whereas deamination of a N⁴-methylcytosine (4 mC) to 4 mC-NO or a 5-methylcytosine (5 mC) to a T causes the base to be read by a polymerase as a C or a T, respectively. For 5 mC bases, the C to T ratio at the 5 mC position is about 40% higher than other cytosine positions, allowing 5 mC to be differentiated from C. (See, Li et al. (2022) Genome Biology 23:122.)

In various embodiments, performing the assay includes enriching for specific genomic sequences, such as genomic sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to target (e.g., hybridize with) selected genomic sequences, thereby capturing and enriching the selected genomic sequences.

In various embodiments, performing the assay includes a step of nucleic acid amplification. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected genomic sequences, a PCR assay is performed to amplify the pre-selected genomic sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the genomic sequence of the pre-selected CGI.

In various embodiments, performing the assay includes quantifying the nucleic acids including the pre-selected genomic sequences (e.g., informative CGIs). In some embodiments, quantifying the nucleic acids to generate sequence information comprises performing an enzyme-linked immunosorbent assay (ELISA). In some embodiments, quantifying the nucleic acids to generate sequence information comprises performing quantitative PCR (qPCR) or digital PCR (dPCR). Therefore, the number of methylated, unmethylated, or partially methylated pre-selected genomic sequences can be quantified.

In various embodiments, quantifying the nucleic acids comprises sequencing the nucleic acids including the pre-selected genomic sequences. Thus, the sequenced reads can be aligned to a reference library and methylation sequence information including methylation statuses of the informative CGIs can be determined. Therefore, the number of methylated, unmethylated, or partially methylated pre-selected genomic sequences can be quantified via the sequenced reads.

FIG. 3B shows an example flow process for determining whether an individual is at risk for a cancer, in accordance with an embodiment. Here, specific genomic regions of an indexed library of nucleic acids (e.g., DNA) are targeted. For example, locus 1 can refer to a reference genomic location. Here, a reference genomic location serves as a control. For example, the reference genomic location is not differentially methylated in healthy individuals in comparison to individuals with the cancer. Locus 2 can refer to a pre-selected genomic location, such as a pre-selected informative CGI.

Performing the assay further includes performing nucleic acid amplification (e.g., PCR) to generate marker information. In various embodiments, nucleic acid amplification includes either qPCR or dPCR. This quantifies the number of methylated, unmethylated, or partially methylated sequences at locus 1 (reference) and at locus 2. In various embodiments, performing the assay includes performing an ELISA to quantify the number of methylated, unmethylated, or partially methylated sequences at locus 1 (reference) and at locus 2.

### Assays for Generating Sequencing Information for Performing Intra-Individual Analysis

In particular embodiments, assays disclosed herein (e.g., assay 120A or 120B shown in FIGS. 1A-1B) are useful for generating sequencing information for performing an intra-individual analysis (e.g., one or both of intra-individual analysis 128A and intra-individual analysis 128B shown in FIGS. 1A-1B). For example, an assay is performed to generate sequence information for target nucleic acids and/or reference nucleic acids.

In various embodiments, sequence information of target nucleic acids and/or sequence information of reference nucleic acids refer to statuses for a plurality of genomic sites. Sequence information of target nucleic acids refers to epigenetic statuses (e.g., methylation statuses) across a plurality of genomic sites in the target nucleic acids. Sequence information of reference nucleic acids refers to epigenetic statuses (e.g., methylation statuses) across a plurality of genomic sites in the reference nucleic acids. In various embodiments, the plurality of genomic sites are previously identified and selected. For example, the plurality of genomic sites may be one or more CpG sites whose differential methylation are informative for determining whether an individual has a cancer. A CpG site is portion of a genome that has cytosine and guanine separated by only one phosphate group and is often denoted as “5′-C-phosphate-G-3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” Cancer informative CGI can be a “CGI identifier” or reference number to allow referencing CGIs during data processing by their respective unique CGI identifiers. Example CGIs include, but are not limited to, the CGIs shown in the accompanying tables (referred to herein as Tables 1-4) which lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing such as next generation sequencing or via quantitative methods such as an ELISA, quantitative PCR, allele-specific PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

In various embodiments, performing an assay (e.g., assay 120A or assay 120B) involves processing target nucleic acids and/or reference nucleic acids. In various embodiments, processing target nucleic acids and/or reference nucleic acids to capture methylation modifications includes performing a nucleic acid conversion (e.g., any of bisulfite conversion, enzymatic conversion, or nitrite conversion). In various embodiments, processing target nucleic acids and/or reference nucleic acids to capture methylation modifications includes performing any of nucleic acid amplification, polymerase chain reaction (PCR), methylation specific PCR, bisulfite pyrosequencing, single-strand conformation polymorphism (SSCP) analysis, methylation-sensitive single-strand conformation analysis restriction analysis, high resolution melting analysis, methylation-sensitive single-nucleotide primer extension, restriction analysis, microarray technology, next generation methylation sequencing, nanopore sequencing, and combinations thereof.

In various embodiments, performing the assay includes enriching for specific sequences in the target nucleic acids and/or reference nucleic acids. In various embodiments, the specific sequences refer to sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to hybridize with particular sequences of the target nucleic acids and/or reference nucleic acids, thereby capturing and enriching the particular sequences.

In various embodiments, performing the assay includes performing nucleic acid amplification to amplify the particular sequences of the target nucleic acids and/or reference nucleic acids. Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected sequences, a PCR assay is performed to amplify the pre-selected sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the sequence of the pre-selected CGI.

In various embodiments, performing the assay includes quantifying the nucleic acids including the pre-selected sequences (e.g., informative CGIs). In some embodiments, quantifying the nucleic acids to generate sequence information comprises performing any of real-time PCR assay, quantitative real-time PCR (qPCR) assay, digital PCR (dPCR) assay, allele-specific PCR assay, or reverse-transcription PCR assay. Therefore, the number of methylated, hypermethylated, unmethylated, or partially methylated pre-selected sequences are quantified.

In various embodiments, quantifying the nucleic acids comprises sequencing the nucleic acids including the pre-selected sequences. Thus, the sequenced reads are aligned to a reference library and sequence information including methylation statuses of the informative CGIs of amplicons derived from the target nucleic acids and/or reference nucleic acids can be determined. Therefore, the number of methylated, hypermethylated, unmethylated, or partially methylated pre-selected sequences of the target nucleic acids and the reference nucleic acids can be quantified via the sequenced reads.

### Assays for Generating Sequencing Information for Phased Sequencing

In various embodiments, performing the assay comprises sequencing the target nucleic acids and/or reference nucleic acids. In various embodiments, sequencing comprises performing next generation sequencing methods to generate sequence reads from the target nucleic acids and/or reference nucleic acids. As described herein, sequence reads from reference nucleic acids may be long sequence reads (e.g., greater than 500 bases in length). Generally, long sequence reads include an average read length that is longer than sequence reads obtained through standard sequencing methods. In various embodiments, the long sequence reads of reference nucleic acids refer to sequence reads of at least 500 bases, at least 1 kilobase, at least 2 kilobases (kb), at least 3 kb, at least 4 kb, at least 5 kb, at least 6 kb, at least 7 kb, at least 8 kb, at least 9 kb, at least 10 kb, at least 12 kb, at least 15 kb, at least 20 kb, at least 25 kb, at least 30 kb, at least 40 kb, at least 50 kb, at least 60 kb, at least 70 kb, at least 80 kb, at least 90 kb, at least 100 kb, at least 200 kb, at least 300 kb, at least 400 kb, at least 500 kb, at least 600 kb, at least 700 kb, at least 800 kb, at least 900 kb, at least 1000 kb, at least 1500 kb, or at least 2000 kb. In particular embodiments, the long sequence reads of reference nucleic acids refer to sequence reads of between 5 kb and 100 kb, between 10 kb and 80 kb, between 20 kb and 70 kb, between 30 kb and 60 kb, or between 40 kb and 50 kb. In particular embodiments, long sequence reads of reference nucleic acids refer to sequence reads of greater than about 8 kb, greater than about 9 kb or greater than about 10 kb. In particular embodiments, long sequence reads of reference nucleic acids refer to sequence reads between about 10 kb and about 100 kb, or between about 10 kb and about 2 MB. In various embodiments, generating long sequence reads of reference nucleic acids involves performing nanopore sequencing. Methods for long-read sequencing are known in the art and such methods can be performed using, for example, an Oxford Nanopore instrument (e.g., PromethION™) or Pacific Biosciences Single-Molecule Real-Time (SMRT) sequencing technology.

In various embodiments, performing the assay includes generating phased sequencing information for target nucleic acids and/or reference nucleic acids. As used herein, “phased sequencing information,” also referred to herein as “haplotype sequencing information,” refers to sequencing information derived specifically from a particular source. For example, phased sequencing information or haplotype sequencing information can refer to sequencing information derived from either the maternal or paternal chromosome. Generally, phased sequencing information of target nucleic acids may be useful for determining presence or absence of a cancer because signals originating from the same source (e.g., maternal or paternal chromosome) may provide additional information in comparison to other approaches that merely analyze signals irrespective of the source.

In various embodiments, the phased sequencing information comprises mutation sequence information of the cell-free DNA. For example, mutation sequence information can include one or more mutations present across a plurality of genomic sites. In particular embodiments, the mutation sequence information includes one or more mutations that originate from a common source (e.g., a maternal chromosome or a paternal chromosome). Here, two or more genomic sites derived from a common source that have a particular pattern of mutations (e.g., each having a mutation, some pattern of mutated/non-mutated, or all non-mutated) can be referred to as coupled genomic sites. In various embodiments, a mutation can be any of a single nucleotide polymorphism (SNP), single nucleotide variant (SNV), insertion, deletion, copy number variation (CNV), duplication, or translocation.

In various embodiments, the phased sequencing information comprises methylation sequence information of the cell-free DNA. Methylation sequence information can include methylation statuses across a plurality of genomic sites. In particular embodiments, the methylation sequence information includes methylation statuses of genomic sites from a common source (e.g., a maternal chromosome or a paternal chromosome). As a specific example, methylation at a first genomic site may be coupled with methylation at a second genomic site on the same maternal or paternal chromosome. Two or more genomic sites with a particular methylation pattern (e.g., all methylated, partially methylated, or non-methylated) that originate from the same maternal or paternal chromosome is referred to herein as coupled methylation sites. Example coupled methylation sites may be two or more CGIs disclosed herein (e.g., two or more CGIs disclosed in any of Tables 1-4). In various embodiments, two or more genomic sites of coupled methylation sites may be separated by tens, hundreds, or even thousands of bases. Thus, coupled methylation sites include two or more genomic sites from a common source and need not be limited to genomic sites that are close in proximity (e.g., adjacent CpG sites). In various embodiments, coupled methylation sites include 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more methylation sites from a common source. Thus, detecting these coupled methylation sites may provide disease diagnostic utility.

In various embodiments, generating phased sequencing information for target nucleic acids comprises aligning sequence reads of target nucleic acids to long sequence reads of reference nucleic acids derived from different sources (e.g., either the maternal or paternal chromosome). Long sequence reads of reference nucleic acids originating from different sources can be distinguished due to sequence differences present in the long sequence reads. For example, given a particular chromosome, long sequence reads derived from a maternal chromosome would have sequence differences in comparison to long sequence reads derived from a paternal chromosome. Here, sequence differences can refer to mutations that are present in long sequence reads from one source, but not present in long sequence reads from the second source, and vice versa. Thus, the presence or absence of certain mutations can be useful for distinguishing whether a long sequence read originated from a first source or a second source. Altogether, by comparing sequences of long sequence reads, a first set of long sequence reads with a set of common sequences can be attributed to a first source (e.g., a maternal chromosome) whereas a second set of long sequence reads with a different set of common sequences can be attributed to a second source (e.g., a paternal chromosome). In various embodiments, the different sets of long sequence reads need not specifically be attributed to a maternal chromosome and a paternal chromosome; rather, it is sufficient to distinguish different sets of long sequence reads from a first source and a second source. These long sequence reads from a first source or a second source have sufficiently different sequences to enable phasing of the target nucleic acids (e.g., to determine sources from which target nucleic acids were derived from).

By aligning sequence reads of target nucleic acids to long sequence reads of reference nucleic acids, the long sequence reads of reference nucleic acids serve as digital guides to phase e.g., determine the source of target nucleic acids. For example, target nucleic acids from a first common source (e.g., from a maternal chromosome) can be categorized together based on sequence similarities between the target nucleic acids and the long sequence reads of reference nucleic acids from the first source. Additionally, target nucleic acids from a second common source (e.g., from a paternal chromosome) can be categorized together based on sequence similarities between the target nucleic acids and the long sequence reads of reference nucleic acids from the second source. In contrast to using the standard human genome to align sequence reads of target nucleic acids, using long reads of reference nucleic acids would enable alignment of reference nucleic acids to sequences of the maternal or paternal chromosome Individual-specific differences between target nucleic acids deriving from the maternal and paternal chromosomes could be used as markers to create haplotype-specific sequence information that is informative for determining presence or absence of a cancer.

In various embodiments, phased sequencing information includes phased methylation sequencing information of cfDNA, where at least a first set of the phased methylation sequencing information of cfDNA originates from a first source and at least a second set of the phased methylation sequencing information of cfDNA originates from a second source. In various embodiments, methods for generating phased sequencing information can further include comparing the first set of the phased methylation sequencing information of cfDNA from the first source to the second set of the phased methylation sequencing information of cfDNA from the second source. In particular embodiments, generating phased sequencing information further includes comparing methylation statuses of two or more genomic sites from a first source to methylation statuses of the same two or more genomic sites from a second source. Differences in methylation statuses of genomic sites from the first source and the second source can be valuable for inclusion in the signal informative for determining presence or absence of a cancer. For example, if multiple genomic sites from a first source are methylated but the same genomic sites from a second source are unmethylated, this may be an informative signal for presence or absence of a cancer.

### Screen

The description in this section pertains to the performance of a screen, such as screen 125 described in FIG. 1A, which can be performed by the screen module 210 described in FIG. 2A. Generally, a screen is performed on marker information generated by the assay (e.g., assay 120A). In various embodiments, the screen is performed to determine whether a biological sample is at risk or not at risk of containing a signal indicative of a cancer. For example, the screen is performed to determine whether a biological sample is at risk or not at risk of containing circulating tumor DNA. Circulating DNA within the biological sample may indicate that the individual (e.g., individual from whom the biological sample is obtained) may be at risk of a cancer. In various embodiments, the screen is performed to classify the subject as negative for cancer or not negative for cancer.

In various embodiments, the marker information represents quantified values of biomarkers. For example, depending on the type of biomarker, the quantified values may be generated via one or more of: an immunoassay, a protein-binding assay, an antibody-based assay, an antigen-binding protein-based assay, a protein-based array, an enzyme-linked immunosorbent assay (ELISA), a Western blot, quantitative PCR (qPCR) or digital PCR (dPCR), NMR, mass spectrometry, LC-MS, or UPLC-MS/MS.

In various embodiments, performing the screen involves comparing the quantified values of biomarkers to one or more reference values or to threshold values. For example, a reference value can be a statistical measure of quantified biomarker values corresponding to individuals known to be at risk for cancer. Therefore, if the comparison identifies that the quantified values of biomarkers for an individual is statistically significantly different from the reference value corresponding to individuals known to be at risk for cancer, then the screen can identify the cancer as negative for cancer.

In various embodiments, the marker information represents sequencing information for one or more genomic locations, such as one or more CpG islands. In various embodiments, performing the screen involves comparing methylation information at one or more pre-selected genomic locations to quantified values of reference genomic locations. For example, referring again to FIG. 3B, an assay may have been performed that generates methylation information for locus 1 corresponding to a reference genomic location and for locus 2 corresponding to a pre-selected genomic location (e.g., a pre-selected informative CGI). Thus, the methylation information at locus 1 is compared to methylation information at locus 2. Based on the comparison, the screen can identify the subject as not negative for cancer.

In various embodiments, the screen can be a cheaper and less complex test in comparison to the second tier analysis (e.g., the second analysis). The screen can analyze marker information at a low resolution for purposes of identifying and removing large proportions of individuals that are not at risk of cancer. In various embodiments, the screen analyzes methylation information across a plurality of genomic locations and determines a measure of overall methylation across the plurality of genomic locations. Here, the measure of overall methylation across the plurality of genomic sites can represent methylation information of low resolution. Specifically, the measure of overall methylation provides a metric for methylation across the plurality of genomic sites, but may not provide information as to methylation status at each individual genomic site. The measure of overall methylation can be sufficient for identifying and removing large proportions of individuals not at risk for cancer. In various embodiments, the overall methylation across the plurality of genomic sites can be a total number of methylated CpG sites. In various embodiments, the overall methylation across the plurality of genomic sites can be a total number of methylated CpG sites across the plurality of genomic sites located in a subset of the CGIs in any one of Tables 1, 2, 3, or 4. In various embodiments, the overall methylation across the plurality of genomic sites can be a total number of methylated CpG sites across the plurality of genomic sites located in all of the CGIs in any one of Tables 1, 2, 3, or 4. In various embodiments, the overall methylation across the plurality of genomic sites can be an average number of methylated CpG sites (e.g., an average number of methylated CpG sites within a target region or a CGI). In various embodiments, the overall methylation across the plurality of genomic sites can be an average number of methylated CpG sites across the plurality of genomic sites located in a subset of the CGIs in any one of Tables 1, 2, 3, or 4. In various embodiments, the overall methylation across the plurality of genomic sites can be an average number of methylated CpG sites across the plurality of genomic sites located in all of the CGIs in any one of Tables 1, 2, 3, or 4.

In various embodiments, performing the screen involves performing whole genome sequencing or whole genome bisulfite sequencing and determining the overall methylation across the whole genome. Thus, in such embodiments, performing the screen is not limited to only analyzing CGIs or portions thereof; rather, performing the screen involves analyzing methylation statuses across the whole genome. In various embodiments, analyzing the methylation statuses across the whole genome can involve determining a quantifiable measure of the overall methylation across the whole genome. In various embodiments, the quantifiable measure of overall methylation is a score, such as a whole genome methylation burden score. In various embodiments, the higher the whole genome methylation burden score, the more likely the biological sample is at risk for containing circulating tumor DNA. In various embodiments, the lower the whole genome methylation burden score, the less likely the biological sample is at risk for containing circulating tumor DNA. In various embodiments, the biological sample is classified as negative (e.g., not at risk for containing circulating tumor DNA) or not negative (e.g., at risk for containing circulating tumor DNA) based on the determined whole genome methylation burden score. For example, if the whole genome methylation burden score for the biological sample is above a threshold score, the biological sample can be classified as not negative. As another example, if the whole genome methylation burden score for the biological sample is below a threshold score, the biological sample can be classified as negative.

In various embodiments, the measure of overall methylation across one or more pre-selected genomic locations and methylation information for reference genomic locations can be a cycle threshold (Ct) value. Cycle threshold refers to the number of PCR cycles needed for a sample to amplify and cross a threshold. In various embodiments, if a difference between the Ct value of the methylation sequences of the pre-selected genomic locations and the Ct value of the reference genomic locations is greater than a threshold, then the screen identifies the subject as not negative for cancer. If a difference between the Ct value of the methylation sequences of the pre-selected genomic locations and the Ct value of the reference genomic locations is less than a threshold, then the screen identifies the subject as negative for cancer.

In various embodiments, a screen is performed on sequence information generated via sequencing (e.g., next generation sequencing) of sequences at the one or more genomic locations, such as one or more CpG islands. In various embodiments, such a screen is performed using a system comprising a computer storage and a processing system. The screen can further involve the implementation of a machine learning model. For example, the computer storage can store sequence information corresponding to a processed sample, the processed sample including cell-free DNA fragments originating from a liquid biopsy of an individual and having been processed to enrich for cancer informative CGIs, the sequencer information comprising, for each sequenced cell-free DNA fragment corresponding to the cancer informative CGIs, a respective position on the genome for the cell-free DNA fragment and methylation information for the cell-free DNA fragment. The processing system can compute values of the cancer informative CGIs for the individual and applies the values as input to a trained machine learning model. The machine learning model provides a predicted output as to whether the individual is at risk for cancer based on the values of the cancer informative CGIs.

In various embodiments, performing the screen involves analyzing a plurality of CGIs. For example, performing the screen involves analyzing methylation statuses of a plurality of CGIs. Cancer informative CGI can be a “CGI identifier” or reference number to allow referencing CGIs during data processing by their respective unique CGI identifiers. The accompanying tables (e.g., Tables 1-4) lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In some embodiments, performing the screen involves analyzing a plurality of CGIs including one or more CGIs that are methylated in the genome of extraembryonic ectoderm (ExE). Here, such example CGIs may be differentially methylated in the genome of ExE and not methylated in corresponding epiblast or adult tissue. Example CGIs that are methylated in the genome of ExE are further disclosed in Table 3 of WO2022133315, which is hereby incorporated by reference in its entirety.

In various embodiments, performing the screen involves analyzing all of the CGIs in any one of Tables 1, 2, 3, or 4. In various embodiments, performing the screen involves analyzing at most 10% of the CGIs in Table 1. In various embodiments, performing the screen involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 1. In various embodiments, performing the screen involves analyzing at most 10% of the CGIs in Table 2. In various embodiments, performing the screen involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 2. In various embodiments, performing the screen involves analyzing at most 10% of the CGIs in Table 3. In various embodiments, performing the screen involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 3. In various embodiments, performing the screen involves analyzing at most 10% of the CGIs in Table 4. In various embodiments, performing the screen involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 4. In various embodiments, performing the screen involves analyzing at most 10% of the CGIs in Tables 2 and 3. In various embodiments, performing the screen involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Tables 2 and 3.

In various embodiments, performing the screen involves analyzing 1 CGI, 2 CGIs, 3 CGIs, 4 CGIs, 5 CGIs, 6 CGIs, 7 CGIs, 8 CGIs, 9 CGIs, 10 CGIs, 11 CGIs, 12 CGIs, 13 CGIs, 14 CGIs, 15 CGIs, 16 CGIs, 17 CGIs, 18 CGIs, 19 CGIs, 20 CGIs, 21 CGIs, 22 CGIs, 23 CGIs, 24 CGIs, 25 CGIs, 26 CGIs, 27 CGIs, 28 CGIs, 29 CGIs, 30 CGIs, 31 CGIs, 32 CGIs, 33 CGIs, 34 CGIs, 35 CGIs, 36 CGIs, 37 CGIs, 38 CGIs, 39 CGIs, 40 CGIs, 41 CGIs, 42 CGIs, 43 CGIs, 44 CGIs, 45 CGIs, 46 CGIs, 47 CGIs, 48 CGIs, 49 CGIs, or 50 CGIs (e.g., CGIs as shown in any of Tables 1-4 or portions of CGIs shown in any of Tables 1-4). In various embodiments, performing the screen involves analyzing at most 2 CGIs, at most 5 CGIs, at most 10 CGIs, at most 15 CGIs, at most 20 CGIs, at most 25 CGIs, at most 30 CGIs, at most 35 CGIs, at most 40 CGIs, at most 45 CGIs, or at most 50 CGIs (e.g., CGIs as shown in any of Tables 1-4 or portions of CGIs shown in any of Tables 1-4). In various embodiments, performing the screen involves analyzing at most 50 CGIs, at most 100 CGIs, at most 150 CGIs, at most 200 CGIs, at most 300 CGIs, at most 400 CGIs, at most 500 CGIs, at most 600 CGIs, at most 700 CGIs, at most 800 CGIs, at most 900 CGIs, at most 1000 CGIs, at most 1500 CGIs, at most 2000 CGIs, at most 2500 CGIs, at most 3000 CGIs, at most 3500 CGIs, at most 4000 CGIs, at most 4500 CGIs, at most 5000 CGIs, at most 5500 CGIs, or at most 6000 CGIs (e.g., CGIs as shown in any of Tables 1-4 or portions of CGIs shown in any of Tables 1-4). In particular embodiments, performing the screen involves analyzing at most 500 CGIs.

In various embodiments, the screen achieves at least 60% sensitivity in detecting presence of a cancer. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the screen achieves at least 75% sensitivity. In particular embodiments, the screen achieves at least 76% sensitivity. In particular embodiments, the screen achieves at least 77% sensitivity. In particular embodiments, the screen achieves at least 78% sensitivity. In particular embodiments, the screen achieves at least 79% sensitivity. In particular embodiments, the screen achieves at least 80% sensitivity.

In various embodiments, the screen achieves at least 60% specificity in excluding individuals without cancer. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the screen achieves at least 90% specificity. In particular embodiments, the screen achieves at least 91% specificity. In particular embodiments, the screen achieves at least 92% specificity. In particular embodiments, the screen achieves at least 93% specificity. In particular embodiments, the screen achieves at least 94% specificity. In particular embodiments, the screen achieves at least 95% specificity.

In various embodiments, the screen achieves at least 15% positive predictive value. In various embodiments, the screen achieves at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% positive predictive value. In particular embodiments, the screen achieves at least 20% positive predictive value. In particular embodiments, the screen achieves at least 21% positive predictive value. In particular embodiments, the screen achieves at least 22% positive predictive value. In particular embodiments, the screen achieves at least 23% positive predictive value. In particular embodiments, the screen achieves at least 24% positive predictive value. In particular embodiments, the screen achieves at least 25% positive predictive value. In particular embodiments, the screen achieves at least 26% positive predictive value. In particular embodiments, the screen achieves at least 27% positive predictive value. In particular embodiments, the screen achieves at least 28% positive predictive value. In particular embodiments, the screen achieves at least 29% positive predictive value. In particular embodiments, the screen achieves at least 30% positive predictive value. In particular embodiments, the screen achieves at least 31% positive predictive value. In particular embodiments, the screen achieves at least 32% positive predictive value. In particular embodiments, the screen achieves at least 33% positive predictive value. In particular embodiments, the screen achieves at least 34% positive predictive value. In particular embodiments, the screen achieves at least 35% positive predictive value. In particular embodiments, the screen achieves at least 36% positive predictive value. In particular embodiments, the screen achieves at least 37% positive predictive value. In particular embodiments, the screen achieves at least 38% positive predictive value. In particular embodiments, the screen achieves at least 39% positive predictive value. In particular embodiments, the screen achieves at least 40% positive predictive value.

In various embodiments, the screen achieves at least 60% negative predictive value. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the screen achieves at least 95% negative predictive value. In particular embodiments, the screen achieves at least 96% negative predictive value. In particular embodiments, the screen achieves at least 97% negative predictive value. In particular embodiments, the screen achieves at least 98% negative predictive value. In particular embodiments, the screen achieves at least 99% negative predictive value.

### Intra-Individual Analysis

The description in this section pertains to the performance of an intra-individual analysis, such as an intra-individual analysis 128A and/or intra-individual analysis 128B described in FIGS. 1A-1B. In general, the intra-individual analyses are conducted for subjects that were previously determined (e.g., via screen 125 as shown in FIG. 1A) as not negative for cancer. The intra-individual analysis removes baseline biological signatures that are specific for a subject to generate a background-corrected signal. Thus, the second analysis involves analyzing the background-corrected signal to determine whether the individual has cancer.

In various embodiments, an intra-individual analysis is conducted using a single sample, such as a blood sample. The sample may contain target nucleic acids and reference nucleic acids. Target nucleic acids may include signatures that are informative of determining presence or absence of a cancer, and can further include baseline biological signatures. Here, target nucleic acids in the blood sample may be derived from a diseased cell which is associated with the cancer. For example, target nucleic acids can include cell-free DNA in the blood that originates from a diseased cell. In particular embodiments, target nucleic acids are cell-free DNA in the blood that originates from a cancer cell. Reference nucleic acids in the sample refer to nucleic acids that contain baseline biological signatures of the individual. For example, baseline biological signatures of the individual may be present in nucleic acids irrespective of whether the nucleic acids originate from a diseased source, or a non-diseased source. The baseline biological signatures of the reference nucleic acids are generally less informative for determining presence or absence of a cancer in comparison to the informative signatures present in the target nucleic acids. In various embodiments, reference nucleic acids refer to cellular genomic DNA derived from a healthy cell from the individual. In various embodiments, reference nucleic acids found in the sample derive from a cell in a healthy organ of the individual. Example organs include the brain, heart, thorax, lung, abdomen, colon, cervix, pancreas, kidney, liver, muscle, lymph nodes, esophagus, intestine, spleen, stomach, and gall bladder. In particular embodiments, reference nucleic acids are found in the sample and refer to cellular genomic DNA derived from peripheral blood mononuclear cells (PBMCs) (e.g., lymphocytes or monocytes) or polymorphonuclear cells (e.g., eosinophils or neutrophils).

In various embodiments, target nucleic acids and reference nucleic acids are separately obtained from the single sample. In various embodiments, the sample is processed to separate the target nucleic acids and reference nucleic acids. For example, the sample may be processed through any one of centrifugation, filtration, gel electrophoresis, bead capture, or matrix extraction. In particular embodiments, target nucleic acids are cell-free nucleic acids and therefore, can be obtained from the supernatant of the separated sample. In particular embodiments, reference nucleic acids are cellular genomic nucleic acids and therefore, can be obtained from a different portion of the separated sample that contains cells.

Generally, an intra-individual analysis is performed on sequence information of target nucleic acids and sequence information of reference nucleic acids. In particular embodiments, the sequence information of target nucleic acids comprise sequence information of cell free DNA. In particular embodiments, the sequence information of reference nucleic acids comprise sequence information of cells, such as peripheral blood mononuclear cells (PBMCs) or polymorphonuclear cells.

The intra-individual analysis involves combining the sequence information of target nucleic acids and sequence information of reference nucleic acids to generate a background-corrected signal informative for determining presence or absence of a cancer. In various embodiments, combining the sequence information of target nucleic acids and sequence information of reference nucleic acids involves differentiating between signatures present or absent in the sequence information of target nucleic acids and signatures present or absent in the sequence information of the reference nucleic acids. For example, if particular signatures are present in the sequence information of target nucleic acids, and the signatures are also present in the sequence information of reference nucleic acids, the signatures in both the target nucleic acids and reference nucleic acids may represent baseline biological signatures. Thus, these signatures may be excluded from the resulting signal informative of determining presence or absence of the cancer. As another example, if particular signatures are present in the sequence information of target nucleic acids, but those signatures are absent in the sequence information of reference nucleic acids, the signatures may not be baseline biological signatures. Thus, these signatures may be included in the resulting signal informative of determining presence or absence of the cancer.

In various embodiments, combining the sequence information of the target nucleic acids and the sequence information of the reference nucleic acids includes aligning the sequence information of the target nucleic acids and the sequence information of the reference nucleic acids. For example, aligning the sequence information involves aligning sequences of a plurality of pre-selected genomic sites for the target nucleic acids and sequences of the same or overlapping plurality of pre-selected genomic sites for the reference nucleic acids.

In various embodiments, both the sequence information of the target nucleic acids and the sequence information of the reference nucleic acids are aligned to a reference genome library (e.g., a reference assembly) with known sequences. Therefore, sequence information of the target nucleic acids are aligned to the sequence information of the reference nucleic acids via the reference genome library. In various embodiments, the sequence information of the target nucleic acids is aligned directly with the sequence information of the reference nucleic acids. In such embodiments, a reference genome library need not be used.

In various embodiments, combining the sequence information of the target nucleic acids and the sequence information of the reference nucleic acids includes determining a difference between the sequence information of the target nucleic acids to the sequence information of the reference nucleic acids.

In various embodiments, differences between the sequence information of the target nucleic acids and the sequence information of the reference nucleic acids are performed on a per-position basis. For example, at a first position of a genomic site, the difference between the sequence information of the target nucleic acids at the first position and the sequence information of the reference nucleic acid at the same first position is determined. The process can then be further repeated for additional positions (e.g., for additional positions across the plurality of genomic sites). In various embodiments, the differences are determined on a per-position basis if the sequence information of the target nucleic acids and reference nucleic acids were generated using a sequencing assay (e.g., next generation sequencing) which provides base-level resolution of the sequences.

In various embodiments, differences between the sequence information of the target nucleic acids and the sequence information of the reference nucleic acids are performed on a per-CGI basis. For example, at a first CGI of a genomic site, the difference between the sequence information of the target nucleic acids at the first CGI and the sequence information of the reference nucleic acid at the same CGI or overlapping portion of the first CGI is determined. The process can then be further repeated for additional CGIs (e.g., for additional CGIs across the plurality of genomic sites). In various embodiments, the differences are determined on a per-CGI basis if the sequence information of the target nucleic acids and reference nucleic acids were generated using a quantitative assay (e.g., qPCR assay).

In various embodiments, differences between the sequence information of the target nucleic acids and the sequence information of the reference nucleic acids are performed on a per-allele basis. For example, at a first allele of a genomic site, the difference between the sequence information of the target nucleic acids at the first allele and the sequence information of the reference nucleic acid at the same allele or overlapping portion of the first allele is determined. The process can then be further repeated for additional alleles (e.g., for additional alleles across the plurality of genomic sites). In various embodiments, the differences are determined on a per-allele basis if the sequence information of the target nucleic acids and reference nucleic acids were generated using a quantitative assay (e.g., qPCR assay or allele-specific PCR assay).

In various embodiments, the intra-individual analysis generates a background-corrected signal that comprises phased sequencing information. As described herein, phased sequence information is derived specifically from a particular source and therefore, may be useful for determining presence or absence of a cancer because signals originating from the same source (e.g., maternal or paternal chromosome) may provide additional information in comparison to other approaches that merely analyze signals irrespective of the source. In various embodiments, performing the intra-individual analysis includes removing baseline biological signatures that would otherwise have been interpreted as being derived from a particular source. As described herein, phased sequencing information can include coupled genomic sites and/or coupled methylation sites from common sources. Therefore, by performing the intra-individual analysis, the coupled genomic sites and/or coupled methylation sites can be informative signatures deriving from common sources as opposed to baseline biological signatures.

Reference is now made to FIG. 3C, which depicts an example combining of sequence information of target nucleic acids and reference nucleic acids to generate a signal informative for a cancer, in accordance with an embodiment. The sequence information of the target nucleic acids and the sequence information of the reference nucleic acids include methylation statuses across a plurality of genomic sites. FIG. 3C shows an example genomic site in which nucleotide bases may be differentially methylated in the target nucleic acid and the reference nucleic acid. For example, as shown in FIG. 3C, the nucleotide base at the second position is methylated (as represented by the presence of a cytosine base which arises following bisulfite conversion) in both the target nucleic acid and the reference nucleic acid. Given that the methylation at the second position occurs in both the target nucleic acid and the reference nucleic acid, this may be a baseline biological signature. Conversely, the target nucleic acid may additionally be methylated at the sixth position and the ninth position, whereas the reference nucleic acid is unmethylated at the sixth position and the ninth position. Here, given that the reference nucleic acid is not methylated at the sixth and ninth position, the presence of the methylated nucleotide bases in the target nucleic acid may represent signatures that are informative of presence or absence of the cancer. Additionally, at the eleventh nucleotide position, the target nucleic acid is unmethylated whereas the reference nucleic acid is methylated. Here, the methylation of the reference nucleic acid can be interpreted as a baseline biological signature.

The differences between the methylation status at each position of the target nucleic acid and the reference nucleic acid can represent the cancer signal. As shown in FIG. 3C, the cancer signal includes methylation statuses at the genomic site, wherein the sixth and ninth position are methylated. Thus, the cancer signal includes signatures from the target nucleic acids that are likely informative of the cancer (e.g., methylated statuses of the sixth and ninth nucleotide bases), and further excludes baseline biological signatures (e.g., baseline biological signatures present in reference nucleic acids such as methylated statuses of the second and eleventh nucleotide bases).

### Second Analysis

The description in this section pertains to the performance of a second analysis, such as second analysis 130 described in FIG. 1A, which can be performed by the second analysis module 220 described in FIG. 2A. Generally, a second analysis is performed on sequence information generated by the assay (e.g., assay 120A or assay 120B). In various embodiments, the second analysis is performed to determine whether a biological sample obtained from an individual contains a signal indicative of a cancer. For example, the screen is performed to determine whether a biological sample contains circulating tumor DNA. Circulating DNA within the biological sample may indicate that the individual (e.g., individual from whom the biological sample is obtained) has cancer. In various embodiments, the second analysis is performed on background-corrected methylation information from an intra-individual analysis to classify the subject as having cancer or not having cancer. In various embodiments, the second analysis is performed to analyze a change in background-corrected methylation information from two or more intra-individual analyses. By analyzing a change in background-corrected methylation information, the second analysis can predict a change in tumor heterogeneity e.g., for tracking tumor heterogeneity in the subject for guided therapy.

In various embodiments, a second analysis is performed on background-corrected sequence information generated via sequencing (e.g., next generation sequencing) of sequences at the one or more genomic locations, such as one or more CpG islands. In various embodiments, the background-corrected sequence information is generated as a result of whole genome sequencing and therefore, a second analysis is performed on sequences of one or more genomic locations across the whole genome.

Generally, the second analysis is a more expensive and/or a more complex test in comparison to the first tier (e.g., screen). By implementing a more complex second analysis, the second analysis can achieve a higher positive predictive value than the first tier. In various embodiments, performing the second analysis involves analyzing methylation information across a plurality of genomic locations that represents a higher resolution in comparison to the lower resolution information analyzed in the first tier. For example, the second analysis may determine a high resolution measure of methylation across the plurality of genomic sites that distinguishes individuals having cancer from other individuals not having cancer in accordance with a high performance metric (e.g., high PPV or high sensitivity). Here, the high resolution measure of methylation can provide information as to methylation status at each individual genomic site and/or methylation statuses across a group of genomic sites.

In various embodiments, the high resolution measure of methylation can be a total quantity of consecutively methylated CpG sites within target regions. In some embodiments, the high resolution measure of methylation can be a total quantity of 3 consecutively methylated CpG sites (referred to as “K3N3”) within target regions. In some embodiments, the high resolution measure of methylation can be a total quantity of 4 consecutively methylated CpG sites (referred to as “K4N4”) within target regions. In some embodiments, the high resolution measure of methylation can be a total quantity of 5 consecutively methylated CpG sites (referred to as “K5N5”) within target regions. For example, the high resolution measure of methylation can be a total quantity of 3, 4, 5, 6, 7, 8, 9, or 10 consecutively methylated CpG sites within a subset of the CGIs in any one of Tables 1, 2, 3, or 4. As another example, the high resolution measure of methylation can be a total quantity of 3, 4, 5, 6, 7, 8, 9, or 10 consecutively methylated CpG sites within all of the CGIs in any one of Tables 1, 2, 3, or 4. In some embodiments, the high resolution measure of methylation can be a proportion of 3 consecutively methylated CpG sites (referred to as “K3N3”) within target regions. In some embodiments, the high resolution measure of methylation can be a proportion of 4 consecutively methylated CpG sites (referred to as “K4N4”) within target regions. In some embodiments, the high resolution measure of methylation can be a proportion of 5 consecutively methylated CpG sites (referred to as “K5N5”) within target regions. For example, the high resolution measure of methylation can be a proportion of 3, 4, 5, 6, 7, 8, 9, or 10 consecutively methylated CpG sites within a subset of the CGIs in any one of Tables 1, 2, 3, or 4. As another example, the high resolution measure of methylation can be a proportion of 3, 4, 5, 6, 7, 8, 9, or 10 consecutively methylated CpG sites within all of the CGIs in any one of Tables 1, 2, 3, or 4.

In some embodiments, the high resolution measure of methylation can be a total quantity of consecutively methylated CpG sites within one or more CGIs that are methylated in the genome of extraembryonic ectoderm (ExE). Here, such example CGIs may be differentially methylated in the genome of ExE and not methylated in corresponding epiblast or adult tissue. Example CGIs that are methylated in the genome of ExE are further disclosed in Table 3 of WO2022133315, which is hereby incorporated by reference in its entirety.

In various embodiments, the high resolution measure of methylation can include methylation statuses of a plurality of CpG sites from a haplotype (e.g., inherited from either a maternal or paternal source). In various embodiments, the high resolution measure of methylation refers to methylation statuses of at least a portion of the CpGs within a CGI within at least a portion of one or more regions in Tables 1-4 from a common haplotype. In various embodiments, the high resolution measure of methylation refers to methylation statuses of all CpGs within a CGI within at least a portion of one or more regions in Tables 1-4 from a common haplotype. In various embodiments, the high resolution measure of methylation refers to methylation statuses of all CpGs within a CGI within one or more regions in Tables 1-4 from a common haplotype.

In various embodiments, the second analysis is performed using a system comprising a computer storage and a processing system. The second analysis can involve the implementation of trained machine learning models, details of which are described in further detail herein. For example, the computer storage can store sequence information corresponding to a processed sample, the processed sample including cell-free DNA fragments originating from a liquid biopsy of an individual and having been processed to enrich for cancer informative CGIs, the sequencer information comprising, for each sequenced cell-free DNA fragment corresponding to the cancer informative CGIs, a respective position on the genome for the cell-free DNA fragment and methylation information for the cell-free DNA fragment.

In particular embodiments, the second analysis further reveals, for individuals who are determined to have the cancer, a tissue of origin of the cancer. The second analysis may identify a tissue of origin of the cancer according to the methylation statuses of the cancer informative CGIs. For example, particular methylation patterns across the cancer informative CGIs are attributable to certain tissues, examples of which include the nervous tissue (e.g., brain, spinal cord, nerves), muscle tissue (cardiac muscle, smooth muscle, skeletal muscle), epithelial tissue (e.g., GI tract lining, skin), and connective tissue (e.g., fat, bone, tendon, and ligaments). As a particular example, in patients with brain cancer, a first set of CGIs may be frequently methylated. Therefore, if a similar methylation pattern is observed across the first set of CGIs for an individual who is under analysis, the second analysis can identify that the individual has cancer, and furthermore, that the cancer is localized to the brain.

In various embodiments, the second analysis involves analyzing a plurality of CGIs. For example, the second analysis involves analyzing methylation statuses of a plurality of CGIs. Cancer informative CGI can be a “CGI identifier” or reference number to allow referencing CGIs during data processing by their respective unique CGI identifiers. The accompanying tables (e.g., Tables 1-4) lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In various embodiments, the second analysis involves analyzing all of the CGIs in any one of Tables 1, 2, 3, or 4. In various embodiments, the second analysis involves analyzing at least 10% of the CGIs in Table 1. In various embodiments, the second analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 1. In various embodiments, the second analysis involves analyzing at least 10% of the CGIs in Table 2. In various embodiments, the second analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 2. In various embodiments, the second analysis involves analyzing at least 10% of the CGIs in Table 3. In various embodiments, the second analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 3. In various embodiments, the second analysis involves analyzing at least 10% of the CGIs in Table 4. In various embodiments, the second analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Table 4. In various embodiments, the second analysis involves analyzing at least 10% of the CGIs in Tables 2 and 3. In various embodiments, the second analysis involves analyzing at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the CGIs in Tables 2 and 3.

In various embodiments, the second analysis involves analyzing at least 100 CGIs (e.g., CGIs as shown in any of Tables 1-4). In various embodiments, the second analysis involves analyzing at least 100 CGIs, at least 150 CGIs, at least 200 CGIs, at least 300 CGIs, at least 400 CGIs, at least 500 CGIs, at least 600 CGIs, at least 700 CGIs, at least 800 CGIs, at least 900 CGIs, at least 1000 CGIs, at least 1500 CGIs, at least 2000 CGIs, at least 2500 CGIs, at least 3000 CGIs, at least 3500 CGIs, at least 4000 CGIs, at least 4500 CGIs, at least 5000 CGIs, at least 5500 CGIs, or at least 6000 CGIs (e.g., CGIs as shown in any of Tables 1-4). In particular embodiments, performing the screen involves analyzing at least 500 CGIs. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In various embodiments, the second analysis involves analyzing more CGIs in comparison to the quantity of CGIs analyzed during the screen. For example, the CGIs analyzed during the screen can represent a subset of the CGIs analyzed during the second analysis. In some scenarios, every CpG island analyzed during the screen is further analyzed when performing the second analysis. Therefore, the second analysis represents a more robust and rigorous analysis in comparison to the more rapid and cost-effective screen. In various embodiments, the second analysis involves analyzing at least 2 times the number of CGIs analyzed during the screen. In various embodiments, the second analysis involves analyzing at least 3 times, at least 4 times, at least 5 times, at least 6 times, at least 7 times, at least 8 times, at least 9 times, at least 10 times, at least 11 times, at least 12 times, at least 13 times, at least 14 times at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, or at least 40 times the number of CGIs analyzed during the screen. In particular embodiments, the second analysis involves analyzing at least 5 times the number of CGIs analyzed during the screen. For example, the screen may involve analyzing at least 100 CGIs and the second analysis may involve analyzing at least 500 CGIs.

In various embodiments, the second analysis achieves at least 60% sensitivity in detecting presence of a cancer. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the second analysis achieves at least 85% sensitivity. In particular embodiments, the second analysis achieves at least 86% sensitivity. In particular embodiments, the second analysis achieves at least 87% sensitivity. In particular embodiments, the second analysis achieves at least 88% sensitivity. In particular embodiments, the second analysis achieves at least 89% sensitivity. In particular embodiments, the second analysis achieves at least 90% sensitivity.

In various embodiments, the second analysis achieves at least 60% specificity in excluding individuals without the cancer. In various embodiments, the second analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the second analysis achieves at least 90% specificity. In particular embodiments, the second analysis achieves at least 91% specificity. In particular embodiments, the second analysis achieves at least 92% specificity. In particular embodiments, the second analysis achieves at least 93% specificity. In particular embodiments, the second analysis achieves at least 94% specificity. In particular embodiments, the second analysis achieves at least 95% specificity.

In various embodiments, the second analysis achieves at least 60% positive predictive value. In various embodiments, the second analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In particular embodiments, the second analysis achieves at least 80% positive predictive value. In particular embodiments, the second analysis achieves at least 81% positive predictive value. In particular embodiments, the second analysis achieves at least 82% positive predictive value. In particular embodiments, the second analysis achieves at least 83% positive predictive value. In particular embodiments, the second analysis achieves at least 84% positive predictive value. In particular embodiments, the second analysis achieves at least 85% positive predictive value.

In various embodiments, the second analysis achieves at least 60% negative predictive value. In various embodiments, the second analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the second analysis achieves at least 90% negative predictive value. In particular embodiments, the second analysis achieves at least 91% negative predictive value. In particular embodiments, the second analysis achieves at least 92% negative predictive value. In particular embodiments, the second analysis achieves at least 93% negative predictive value. In particular embodiments, the second analysis achieves at least 94% negative predictive value. In particular embodiments, the second analysis achieves at least 95% negative predictive value. In particular embodiments, the second analysis achieves at least 96% negative predictive value. In particular embodiments, the second analysis achieves at least 97% negative predictive value. In particular embodiments, the second analysis achieves at least 98% negative predictive value. In particular embodiments, the second analysis achieves at least 99% negative predictive value.

### Longitudinal Analysis

In various embodiments, methods disclosed herein are valuable for performing longitudinal analysis for a subject. For example, a subject who was determined to have a presence of cancer (e.g., through the screen or through the second analysis) can be further tracked through a longitudinal analysis. In various embodiments, an additional sample is obtained from the subject at a subsequent timepoint, and the second analysis can be further performed for the subject using the additional sample. Thus, the second analysis performed for the additional sample can determine a change in the cancer for the subject over the intervening timeframe.

In various embodiments, a longitudinal analysis can be performed for subjects who may have been identified as not having cancer. In various embodiments, a longitudinal analysis is performed for subjects who were identified as negative through the screen (e.g., first analysis). In various embodiments, a longitudinal analysis is performed for subjects who were identified as negative through the second analysis. In various embodiments, a longitudinal analysis is performed for subjects who were identified as not negative through the screen and then further identified as negative through the second analysis. By longitudinally tracking subjects who may have been identified as not having cancer, any false negative subjects can potentially be identified through subsequent testing of one or more additional samples obtained at one or more subsequent timepoints. For example, a subject can be identified as not negative through the screen, and through the longitudinal analysis (e.g., at a subsequent timepoint), an additional sample of the subject can be analyzed using either the methodology described in reference to the screen or the second analysis to identify the subject as a false negative. As another example, a subject can be identified as not negative through the second analysis, and through the longitudinal analysis (e.g., at a subsequent timepoint), an additional sample of the subject can be analyzed using either the methodology described in reference to the screen or the second analysis to identify the subject as a false negative.

Reference is now made to the tumor tracking module 230, which represents a module of the tumor heterogeneity system 170 as shown in FIG. 2A. In various embodiments, tracking tumor heterogeneity over two or more timepoints enables the determination of whether an intervention is efficacious. Given a subject who has previously received the intervention (e.g., a tumor therapeutic) for treating cancer, tracking tumor heterogeneity over two or more timepoints using the methods disclosed herein is informative for determining whether the intervention is efficacious for treating the cancer. Generally, a subject exhibiting a reduction in tumor heterogeneity over two or more timepoints is indicative that the tumor subclones are decreasing and that the intervention is effective. Alternatively, a subject who does not exhibit a reduction in tumor heterogeneity (e.g., stable or increase tumor heterogeneity) is indicative that the tumor subclones is unchanging or is increasing. In this scenario, the intervention lacks efficacy. Thus, methods for tracking tumor heterogeneity can be useful for e.g., guided therapy.

In various embodiments, tracking tumor heterogeneity for a subject comprises obtaining samples from the subject across two or more timepoints, performing intra-individual analysis for one or more of the obtained samples, and generating predictions across at least the two or more timepoint. The predictions can be informative for the subject's tumor heterogeneity. In various embodiments, tracking tumor heterogeneity for a subject comprises obtaining three or more samples from a subject across at least three timepoints, performing intra-individual analysis for the three or more samples, and generating predictions across the at least three timepoints. In various embodiments, tracking tumor heterogeneity for a subject comprises obtaining four or more samples from a subject across at least four timepoints, performing intra-individual analysis for the four or more samples, and generating predictions across the at least four timepoints. In various embodiments, tracking tumor heterogeneity for a subject comprises obtaining samples from a subject, performing intra-individual analysis for each of the obtained samples, and generating predictions across at least five timepoints, at least six timepoints, at least seven timepoints, at least eight timepoints, at least nine timepoints, at least ten timepoints, at least eleven timepoints, at least twelve timepoints, at least thirteen timepoints, at least fourteen timepoints, at least fifteen timepoints, at least sixteen timepoints, at least seventeen timepoints, at least eighteen timepoints, at least nineteen timepoints, or at least twenty timepoints.

In various embodiments, the time between any two timepoints can be between 1 day and 12 months, between 5 days and 8 months, between 10 days and 6 months, between 15 days and 4 months, between 20 days and 3 months, between 30 days and 2 months. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 day and 100 days, between 5 day and 80 days, between 10 days and 70 days, between 15 days and 60 days, between 20 days and 50 days, between 25 days and 40 days, or between 30 days and 35 days. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 month and 2 months.

In various embodiments, methods for tracking tumor heterogeneity involve obtaining a sample from the subject at a first timepoint (e.g., an initial timepoint), performing an intra-individual analysis using the obtained sample, and generating a cancer prediction for the sample obtained at the first timepoint. In various embodiments, the first timepoint may refer to a timepoint prior to which the subject receives an intervention, such as a tumor therapeutic. Thus, the generated for the sample obtained at the first timepoint may represent a baseline prediction prior to any therapeutic treatment. In various embodiments, the first timepoint may refer to a timepoint immediately after the subject receives an intervention, such as a tumor therapeutic. In this context, “immediately after” the subject receives an intervention can refer to a timeframe within 1 day after the subject receives the intervention. In various embodiments, “immediately after” refers to a timeframe within 12 hours, within 8 hours, within 6 hours, within 4 hours, within 3 hours, within 2 hours, within 1 hour, within 30 minutes, within 15 minutes, within 10 minutes, within 5 minutes, or within 1 minute of the subject receiving the therapeutic.

In particular embodiments, methods for tracking tumor heterogeneity further involve obtaining one or more subsequent samples from the subject after the first timepoint (e.g., at a second timepoint, at a third timepoint, at a fourth timepoint, etc.), performing intra-individual analyses for a subsequent sample, and generating predictions for the one or more subsequent samples. In this scenario, the change in the predictions for the one or more subsequent samples in comparison to the prediction of the first sample can be indicative of the change in tumor heterogeneity. In various embodiments, the one or more subsequent samples are obtained from the subject after the subject has received an intervention, such as a tumor therapeutic. Thus, the change in tumor heterogeneity can be reflective of the efficacy, or lack thereof, of the intervention provided to the subject.

### Machine Learning Models for Analyzing Sequence Information

In various embodiments, trained machine learning models can be deployed to analyze sequence information for tracking tumor heterogeneity for a subject across two or more timepoints. In various embodiments, the sequence information includes methylation statuses of plurality of genomic sites. Therefore, trained machine learning models analyze differential methylation of the plurality of genomic sites to output predictions.

In various embodiments, a trained machine learning model is deployed as part of a screen (e.g., screen 125 as shown in FIG. 1A). Thus, the trained machine learning model can analyze sequence information generated via an assay (e.g., assay 120A shown in FIG. 1A) to determine whether a subject is negative or not negative for a cancer. In various embodiments, a trained machine learning model is deployed as part of a second analysis (e.g., second analysis 130 shown in FIG. 1A). Therefore, the trained machine learning model can analyze sequence information including methylation statuses for a plurality of genomic sites, such as a plurality of CpG sites disclosed herein. In various embodiments, the sequence information includes background-corrected sequence information generated via an intra-individual analysis (e.g., intra-individual analysis 128A and/or intra-individual analysis 128B shown in FIG. 1A). In some embodiments, the trained machine learning model analyzes a difference between background-corrected sequence information determined from two intra-individual analyses (as shown in FIG. 1A). In some embodiments, the trained machine learning model analyzes background-corrected sequence information from a single intra-individual analysis (as shown in FIG. 1B).

In various embodiments, a machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In particular embodiments, trained machine learning models analyze methylation statuses of a plurality of genomic sites to generate predictions. The methylation statuses can correspond to a set of cancer informative CpG islands (CGIs), wherein the cancer informative CGIs are selected from a group consisting of a ranked set of candidate CGIs. In various embodiments, a machine learning model analyzes methylation statuses for at least 50 CGIs. In various embodiments, a machine learning model analyzes methylation statuses for at least 100 CGIs. In various embodiments, a machine learning model analyzes methylation statuses for at least 150 CGIs. In various embodiments, a machine learning model analyzes methylation statuses for at least 200 CGIs. In various embodiments, a machine learning model analyzes methylation statuses for at least 250 CGIs. In various embodiments, a machine learning model analyzes methylation statuses for at least 300 CGIs. In various embodiments, a machine learning model analyzes methylation statuses for at least 400 CGIs. In various embodiments, a machine learning model analyzes methylation statuses for at least 500 CGIs. In various embodiments, a machine learning model analyzes methylation statuses for at least 600 CGIs. In various embodiments, a machine learning model analyzes methylation statuses for at least 700 CGIs. In various embodiments, a machine learning model analyzes methylation statuses for at least 800 CGIs. In various embodiments, a machine learning model analyzes methylation statuses for at least 900 CGIs. In various embodiments, a machine learning model analyzes methylation statuses for at least 1000 CGIs. In various embodiments, a machine learning model analyzes methylation statuses for at least 2500 CGIs. In various embodiments, a machine learning model analyzes methylation statuses for at least 5000 CGIs. In various embodiments, a machine learning model analyzes methylation statuses for at least 7500 CGIs. In various embodiments, a machine learning model analyzes methylation statuses for at least 10000 CGIs. In various embodiments, a machine learning model analyzes methylation statuses for at least 15000 CGIs. In various embodiments, a machine learning model analyzes methylation statuses for at least 20000 CGIs. In various embodiments, a machine learning model analyzes methylation statuses for at least 25000 CGIs.

In various embodiments, a machine learning model analyzes methylation statuses for CGIs across the whole genome. For example, a machine learning model may be implemented to analyze sequencing data generated from whole genome sequencing (e.g., whole genome bisulfite sequencing).

Additionally disclosed herein are particular genomic sites, such as CpG islands (CGIs) whose methylation statuses can be informative for determining whether a subject is at risk of a cancer or whether the individual has a cancer. In some embodiments, methylation statuses of the informative CGIs representing a signal in a sample can be indicative of a presence of the cancer. In some embodiments, methylation statuses of the informative CGIs representing a signal in a sample can be indicative of an absence of the cancer. In various embodiments, methods disclosed herein, such as methods involving the multiple-tiered analysis, are useful for detecting or identifying the signal (e.g., methylation statuses of the informative CGIs) in a sample. In various embodiments, methods disclosed herein, such as methods involving the multiple-tiered analysis, are useful for increasing the probability that the detected signal (e.g., methylation statuses of the informative CGIs) in the sample is authentic. A signal (e.g., methylation statuses of the informative CGIs) detected by the multiple-tiered analysis can be confidently trusted as present in the sample. Thus, by tracking the change in methylation statuses for the subject across multiple timepoints, a change in the subject's risk for cancer or a change in the subject's cancer can be more accurately determined.

Methylation statuses of cancer informative CGIs can be useful for predicting whether an individual has a cancer or is at risk for a cancer. In various embodiments, the methylation statuses of cancer informative CGIs are background-corrected methylation statuses of cancer informative CGIs. For example, background-corrected methylation statuses of cancer informative CGIs can be determined via an intra-individual analysis. For example, background-corrected methylation statuses of cancer informative CGIs can be determined by combining methylation information of cancer informative CGIs of target nucleic acids and methylation information of cancer informative CGIs of reference nucleic acids.

In various embodiments, each cancer informative CGI can be a “CGI identifier” or reference number to allow referencing CGIs during data processing by their respective unique CGI identifiers. The accompanying tables (e.g., Tables 1-4) lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

Reference is now made to FIG. 3D, which is an illustrative example of a signal informative for a cancer. In various embodiments, the signal informative for a cancer shown in FIG. 3D can be generated as a result of the intra-individual analysis. Thus, the signal informative for a cancer represents background-corrected sequence information e.g., corrected via an intra-individual analysis that combines sequence information from target nucleic acids and reference nucleic acids. In various embodiments, the signal informative for a cancer shown in FIG. 3D can represent sequence information of target nucleic acids. In such embodiments, the signal is not derived from an intra-individual analysis.

As shown in FIG. 3D, for each instance of an analyte, e.g., a cell-free DNA fragment, there is data indicating, for each of a plurality of positions along the instance of the analyte, e.g., distinct CpG sites along a DNA fragment, information about a marker at that position, e.g., whether that CpG is methylated or unmethylated. An instance of an analyte can be a single sequenced DNA fragment or a portion of a single sequenced DNA fragment. In various embodiments, the DNA fragment may be a bisulfite converted DNA fragment. Therefore, an instance of an analyte may refer to a sequenced bisulfite converted DNA fragment or a portion thereof.

Conceptually, using methylation of CpGs in cell-free DNA as an illustrative example, the signal illustrated in FIG. 3D includes a row, e.g., row 240, for each instance of an analyte, such as a single sequenced DNA fragment. Thus, in FIG. 3D, data for sixteen instances of an analyte are shown, e.g., sixteen DNA fragments. Each circle corresponds to a position along the analyte, such as a CpG site. In this example, whether the circle is illustrated as black or white in FIG. 3D, is indicative of whether the CpG site is methylated (black) or unmethylated (white). In some instances, information about a marker at a position in a nucleic acid may not be binary.

The information about the markers for each instance of an analyte in a sample can result in a large amount of data. As an example, in practice, in the case of obtaining methylation state of CpGs in cell-free DNA from a blood sample using deep sequencing, using a DNA sequencer that outputs such data into a FASTQ format data file, the signal generated by processing a single blood sample can be many gigabytes, e.g., 20 to 30 gigabytes, of data.

FIG. 3D also illustrates a relative alignment among the distinct instances of the analyte. In the example of DNA, for example, the position of a DNA fragment within a genome for the individual from which a sample originated can be determined, and each position within the genome can have a respective set of coordinates identifying it. Thus, DNA fragments can be assigned coordinates based on their respective positions within the genome, and then aligned or grouped by those coordinates. Thus, in FIG. 3D, column 242 indicates a position on an analyte, such as a single CpG site in a genome, and the distinct instances of the analyte are illustrated as aligned by position on the analyte.

By using the position information for each instance of an analyte, distinct instances of the analyte can be grouped into regions within the analyte. Typically, markers related to cancer are localized within identifiable regions of analytes, such as specific genes or regions within the genome. Thus, the signals generated for each instance of an analyte can be grouped and processed by cancer-informative regions. In particular embodiments, an informative region is a CGI (or at least a portion thereof) as disclosed in any of Tables 1-4. The example in FIG. 3D can be considered to illustrate data about methylation at CpG sites within one informative region of the genome, for multiple DNA fragments obtained from a biological sample. There can be multiple cancer-informative regions.

As disclosed herein, trained machine learning models are deployed to generate informative predictions regarding presence or absence of cancer. To use a trained machine learning model in this context, there are several technical problems that arise relating to encoding the signal resulting from processing a biological sample into features. Some problems arise because the signal includes a large amount of information. One of the challenges involves reducing the volume of data into a set of informative features. However, as the number of features increases, the complexity of the computational model increases. However, as the number of features decreases, information relevant to detection of a cancer may be lost. Some problems arise because of uncertainty around which metrics and which regions of an analyte are truly informative of a cancer. Omission of some metrics or some regions from the set of features may adversely impact the performance of a trained computational model.

To address such problems, in various embodiments, very particularly engineered features are generated from a biological sample. Such engineered features may be dependent on one or more health-condition-informative regions (e.g., CGIs) and/or one or more distinct windows within the health-condition informative regions (e.g., CGIs). Each window may have a specified range of positions within a health-condition informative region, and a specified size. The size is specified in terms of a number of consecutive sites of interest within the analyte. A metric is thus computed for a plurality of windows within the health-condition informative region. Thus, in particular embodiments, the engineered features, representing metrics within a particular window within a health-condition informative region (e.g., CGIs), are informative for a cancer.

To train a machine learning model, in some embodiments, a first set of features is computed for a training set, which can include several candidate features. The candidate features can include one or more candidate metrics, or one or more candidate health-condition-informative regions, or combinations of both. A computational model can be trained using candidate features, and then analyzed to determine which candidate features were more influential in the output of the trained computational model. Such analysis can be used to identify features which are more influential to the model, whether due to the metric or due to the health-condition-informative region. A second set of features can be defined by reducing the first set of features based on those identified features which are more influential, and the trained machine learning model can be built using the second set of features.

In various embodiments, to generate data for a machine learning model (e.g., for training or for deployment), the methodology includes computing, for one or more instances of an analyte in a window of a plurality of windows on a target region of the analyte, a metric specific for the window and the target region. The specific metrics used, and health-condition-informative regions selected can depend on a variety of factors and may be experimentally determined. The machine learning model can be implemented to analyze at least the metric specific for the window and the target region. In various embodiments, the metric specific for the window and the target region includes a proportion of a count of DNA fragments having a specific count of methylated CpGs to a count of DNA fragments for the window of the target region. In various embodiments, the metric specific for the window and the target region comprises a proportion of a count of DNA fragments having a specific pattern of methylation to a count of DNA fragments for the window of the target region. As described in further detail below, computing the metric can involve applying two or more functions. For example, computing the metric specific for the window and the target region can involve performing a first function to quantify a count of occurrences of methylated CpGs within the window of the target region. As another example, computing the metric specific for the window and the target region can involve performing a second function to normalize the count of occurrences of methylated CpGs relative to a count of DNA fragments for the window of the target region.

In various embodiments, to generate features, each instance of the analyte (e.g., cell-free DNA) is processed. For each instance of an analyte in the biological sample, and for each window of a plurality of windows on health-condition-informative regions of the analyte, a respective value is generated. After processing instances of the analyte, the feature computation module then computes, for each window of the plurality of windows on the health-condition-informative region, one or more respective metrics for the window based on a first function and/or a second function for instances of the analyte for the window. In various embodiments, a first function quantifies markers within a window. As a specific example, a first function refers to a quantification of a number of methylated CpG sites within a window. In various embodiments, a second function computes a proportion of the quantified markers within the window in relation to other quantified markers. As a specific example, a second function computes the proportion of the number of methylated CpG sites within a window relative to other numbers of methylated CpG sites within a window.

Example implementations will now be described in reference to FIGS. 3E and 3F. Here, in FIG. 3E, illustrative marker information for instances of an analyte are shown schematically for the purposes of simplifying this explanation. In this example, there are ten (10) instances of an analyte, each having a length of six (6) sites of interest, at which marker information is a binary value, indicated by a black or white circle. FIG. 3E shows aligned instances of an analyte, along with the designation of a window with a particular kmer size (e.g., K=3). Each window has a size of three (3) consecutive sites of interest within the analyte. In other embodiments, smaller or larger window sizes may be implemented for the analysis. There are four (4) windows of size three (3) (i.e., a first window that includes the first, second, and third sites of interest from the left, a second window that includes the second, third, and fourth sites of interest from the left, a third window that includes the third, fourth, and fifth sites of interest from the left, and a fourth window that includes the fourth, fifth, and sixth sites of interest from the left), but computations for three (3) windows are shown.

In FIG. 3E an example of a first function applied to an instance of an analyte is a count of occurrences of marker information within the instance of the analyte within the window. For example, where the marker information is methylation of a CpG site, this function can be a count of methylated CpGs in the window. That is, if the window has a size of three sites of interest, then there are four possible counts: 0, 1, 2, and 3. Note that inverse results would be obtained if the count was of unmethylated CpGs in the window, but such results when used in training would have the same effect.

In FIG. 3E, the second function computes counts of the number of instances having each possible count resulting from the first function. That is, if the window has a size of three sites of interest, for which there are four possible counts (0, 1, 2, and 3), for that window the second function computes a count of the number of instances with a count of zero, a count of the number of instances with a count of one, a count of the number of instances with a count of two, and a count of the number of instances with a count of three. The second function divides the respective number of instances computed for possible counts by the total number of instances, thus providing a fractional value for each of the possible counts for this window.

In this example in FIG. 3E, for this health-condition-informative region (referred to as “HCl”), there are windows “W1”, “W2”, and “W3”, each of which has four (4) values, representing the respective count for each possible count of methylated CpGs among the instances that overlap that window. Because there are ten (10) instances, each of these values is divided by 10 in the second function, to provide the respective final four output values for each window. As shown in FIG. 3E, referring to the example of Window 1 (W1), the final four output values are 0.3 (0 methylated CpG sites in the window), 0.1 (1 methylated CpG sites in the window), 0.1 (2 methylated CpG sites in the window), and 0.5 (3 fully methylated CpG sites in the window). Here, the proportion of fully methylated CpG sites, proportion of fully non-methylated CpG sites, and proportion of partially methylated CpG sites (e.g., either 1 or 2 methylated CpG sites in the window) can be metrics informative for a cancer.

Reference is now made to FIG. 3F, which shows an example application of a first function and second function to instances of an analyte. Here, the bottom of FIG. 3F shows patterns of the marker information in the instance, from among a set of possible patterns. A pattern is a unique sequence of marker information along the sites of interest in a window. For example, as shown in FIG. 3F, if the window has a size of three sites of interest, and if the marker information for the sites of information is binary, then there are eight possible patterns. For example, where the marker information is methylation of a CpG site, each possible pattern of methylation in a window is a distinct sequence of the methylation state (e.g., methylated or unmethylated) of the CpG sites along the sequence of consecutive CpG sites in the window. When the marker information is methylation of CpG sites, the first function, applied to an instance of a DNA fragment in a window, outputs an indication of which of the possible patterns of methylation of CpGs is present in the window in that DNA fragment.

The second function computes a count of the number of instances having each possible pattern in a window. That is, for that window, the second function produces a count of the number of instances with the first pattern, a count of the number of instances with the second pattern, and so on. The second function then divides the respective number of instances identified for each possible pattern by the total number of instances, thus providing a fractional value for each of the possible patterns for this window, as shown in the bottom panel of FIG. 3F.

In this example in FIG. 3F, for this health-condition-informative region (say, “HCl”), there are windows “W1”, “W2”, and “W3”, each of which has eight values, representing the respective number of occurrences each possible pattern among the instances that overlap that window divided by the number of instances, in this case ten (10).

In any of the foregoing example implementations, and in other implementations, a size of a health-condition-informative region, in terms of a number of sites of interest within an instance of an analyte, can vary. For example, cancer-informative regions of DNA may be as small as a single CpG site, and may include several 10's, 100's, or 1000's of CpG sites. Within a set of features, there may be a plurality of health-condition-informative regions, each having its own respective size.

In any of the foregoing example implementations, and in other implementations, a size of a window in a health-condition-informative region, in terms of a number of sites of interest within an instance of an analyte, can vary. Generally, the number of sites of interest is a positive integer number that ranges between 1 and N. In some example implementations, N is less than or equal to 10, or 9, or 8, or 7, or 6, or 5, or 4, or 3. In various embodiments, a window within a health-condition informative region includes a specific numbers of CpG sites. In various embodiments, N is 1, 2, 3, 4, 5, 6, 7, 8, 9, or 10 CpG sites. In various embodiments, N is between 1 and 100, between 2 and 80, between 3 and 60, between 4 and 40, between 5 and 20, or between 6 and 10 CpG sites. In various embodiments, N is between 1 and 10, between 2 and 9, between 3 and 8, between 4 and 7, or between 4 and 6 CpG sites. Within a set of features, there may be a plurality of health-condition-informative regions, each having its own respective window size or set of window sizes. Different window sizes may be used in different regions. The same window size may be used in different regions. A region may have metrics computed for it for multiple different window sizes. Windows may be over-lapping or non-overlapping.

In various embodiments, a metric represents an input vector that can be provided as input to a machine learning model (e.g., either during training or deployment of the machine learning model). Here, the metric may be specific for a window and a target region of interest (e.g., a target region comprising one or more CpG sites). For example, the input vector of the metric may include a set of values representing the proportion of counts of methylated CpGs in the window relative to a total count (e.g., total count of DNA fragments for the window of the target region). In various embodiments, the input vector of the metric may include a set of values representing proportions of DNA fragments having specific counts of methylated CpGs out of all possible CpG methylation patterns in the window. The all possible CpG methylation patterns are 2^{k} possible patterns, where k refers to a number of CpG sites in the window. Referring against to the bottom panel of FIG. 3F, an input vector of a metric can be generated for a particular window. Taking the first window (e.g., left-most window shown in FIG. 3F) as an example, the input vector of the metric may include the proportion vales shown in the left most column in the bottom panel of FIG. 3F. Thus, the input vector of the metric may be represented as [0.3, 0.1, 0, 0, 0, 0.1, 0, 0.5]. Similar input vectors for other metrics can be generated using the values of other windows.

The computed sets of values for the set of features for samples can be stored in a data structure, which can be stored in a database, memory, or other computer storage for use in connection with the computational model, or for other purposes.

In some implementations, the sets of values for the set of features for a sample can be stored in association with an identifier of the subject, or an identifier of the sample, or both, so that the identifier of the subject or the identifier of the sample, or both, can be used to access the set of values from the computer storage. In some implementations, each computed value can be associated with an identifier of the cancer-informative region, and an identifier of the window within that region, to which the value corresponds.

Accordingly, an example implementation of such a data structure is shown in FIG. 3G. A set of values for a set of features is stored for a biological sample originating from a subject. The data structure can include an optional identifier for the subject, and an optional identifier for the biological sample. The latter identifier is useful when there are multiple samples for a single subject. For a sample, as indicated at 250, the set of features includes one or more metrics, for each of one or more windows 254, e.g., window “W-1-1”, within each of one or more health-condition-informative regions 252A, e.g., region “R1” or 252B e.g., region “R2”. For each feature, e.g., R-1, W-1-1, Metric, the computed value, e.g., Value 256, is stored. The number of windows in each region can be different for each region. The size of the window can be different for each window. The metric(s) computed for the window can be different for each window.

### Example Methods for Conducting Two or More Intra-Individual Analyses

As disclosed herein, methods involve tracking tumor heterogeneity in a subject by conducting intra-individual analyses for two or more samples obtained from the subject across two or more timepoints. For example, a first intra-individual analysis can be performed for a first sample obtained from the subject at a first timepoint and a second intra-individual analysis can be performed for a second sample obtained from the subject at a second timepoint. Thus, the change in results from each intra-individual analysis can be informative for tracking tumor heterogeneity in the subject.

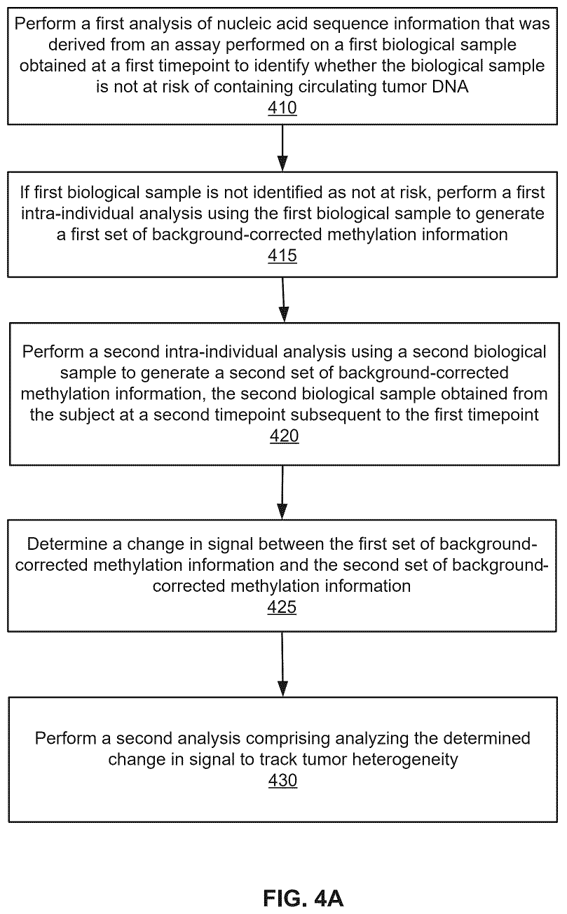

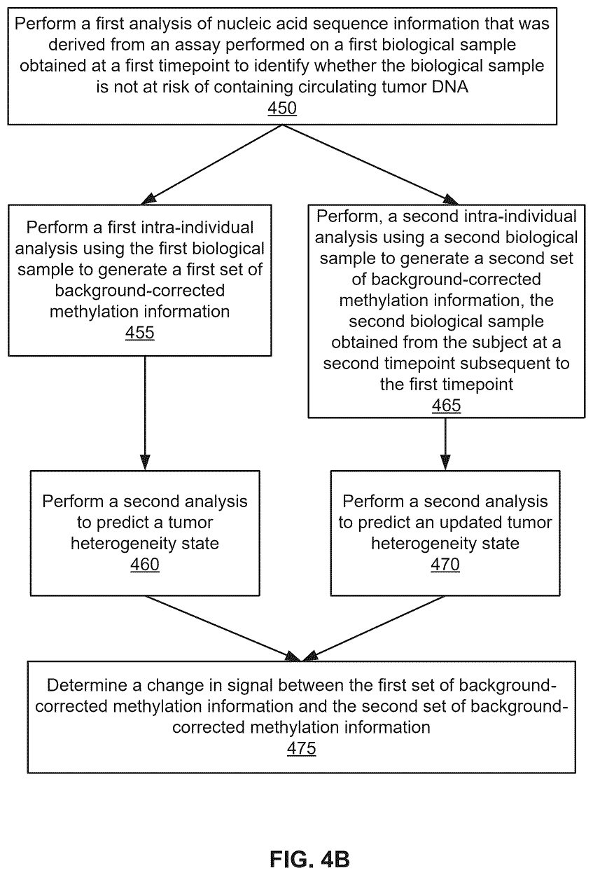

FIG. 4A shows an example flow process involving a first and second intra-individual analyses, in accordance with a first embodiment. In this first embodiment, the flow process involves performing separate intra-individual analyses for first and second samples obtained from the subject at two different timepoints and performing a second analysis on the difference between the results of the separate intra-individual analyses.

Step 410 involves performing a first analysis of nucleic acid sequence information that was derived from an assay performed on a first biological sample obtained at a first timepoint to identify whether the biological sample is not at risk of containing circulating tumor DNA.

Next, at step 415, if the first biological sample is not identified as not at risk, perform a first intra-individual analysis using the first biological sample to generate a first set of background-corrected methylation information.

Step 420 involves performing a second intra-individual analysis using a second biological sample to generate a second set of background-corrected methylation information, the second biological sample obtained from the subject at a second timepoint subsequent to the first timepoint.

Step 425 involves determining a change in signal between the first set of background-corrected methylation information and the second set of background-corrected methylation information.

Step 430 involves performing a second analysis comprising analyzing the determined change in signal to track tumor heterogeneity.

Reference is now made to FIG. 4B, which shows an example flow process involving a first and second intra-individual analyses, in accordance with a second embodiment. In this second embodiment, the flow process involves performing separate intra-individual analyses for first and second samples obtained from the subject at two different timepoints and performing a second analysis on each of the results of the separate intra-individual analyses.

Step 450 involves performing a first analysis of nucleic acid sequence information that was derived from an assay performed on a first biological sample obtained at a first timepoint to identify whether the biological sample is not at risk of containing circulating tumor DNA.

Step 455 involves performing a first intra-individual analysis using the first biological sample to generate a first set of background-corrected methylation information.

Step 460 involves performing a second analysis to predict a tumor heterogeneity state.

Step 465 involves performing a second intra-individual analysis using a second biological sample to generate a second set of background-corrected methylation information, the second biological sample obtained from the subject at a second timepoint subsequent to the first timepoint.

Step 470 involves performing a second analysis to predict an updated tumor heterogeneity state.

Step 475 involves determining a change in signal between the first set of background-corrected methylation information and the second set of background-corrected methylation information.

### Guided Therapy

In various embodiments, the methods disclosed herein for performing a multiple-tiered analysis (e.g., screening and/or intra-individual analysis) to track tumor heterogeneity of one or more cancers in one or more subjects are informative for identifying an intervention for the subject. In various embodiments, an intervention may be any intervention known to those of ordinary skill in the art. Non-limiting examples of interventions include surgery (e.g., excising diseased or pre-disease tissue from an individual), a tumor therapeutic (e.g., chemotherapy, gene therapy, or gene editing), radiation therapy, or a lifestyle intervention (e.g., change in behavior or habits). In particular embodiments, the intervention comprises a tumor therapeutic.

In various embodiments, the methods disclosed herein are performed for a subject who previously received a tumor therapeutic. Thus, tracking the tumor heterogeneity of one or more cancers for the subject can be informative for determining whether the previously provided tumor therapeutic is efficacious. For example, if the tumor heterogeneity of a cancer is not decreasing (e.g., is increasing or is remaining stable) over the two or more timepoints, the tumor therapeutic is deemed non-efficacious. In this example, methods can involve selecting a new intervention, such as a new or different tumor therapeutic, for treatment of the subject's cancer. As another example, if the tumor heterogeneity is decreasing over the two or more timepoints, the tumor therapeutic can be deemed efficacious. In this example, methods can involve selecting the tumor therapeutic that was previously provided to subject. Thus, the tumor therapeutic can continue to be provided to the subject to treat the cancer. In some embodiments, methods can involve selecting a new or different tumor therapeutic for treatment of the subject's cancer. In some embodiments, methods can involve selecting a new or different intervention in addition to the previously provided tumor therapeutic. Thus, the new or different intervention and the previously provided tumor therapeutic can be provided to the subject to treat the cancer.

### Cancers

The disclosure provides methods for performing a multiple-tiered analysis (e.g., screening and/or intra-individual analysis) to track tumor heterogeneity of one or more cancers in one or more subjects. In various embodiments, the subject may have been previously diagnosed with a cancer and receives an intervention for treating the cancer. For example, the subject may have previously received a tumor therapeutic for treating the cancer. In various embodiments, the subject may be suspected of having a cancer, but may not have been previously diagnosed with a cancer. In various embodiments, the subject is healthy and is not yet suspected of having a cancer. In certain embodiments, a cancer is an early-stage health cancer, e.g., prior to development of symptoms.

In various embodiments, the cancer is an early stage cancer. In various embodiments, the cancer is a preclinical phase cancer. In various embodiments, the cancer is a stage I cancer. In various embodiments, the cancer is a stage II cancer. Thus, the methods disclosed herein enable the screening and tracking of tumor heterogeneity of a subject for an early stage or preclinical stage cancer.

In various embodiments, the cancer is any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

### Computer Implementation

The methods of the invention, including the methods of performing a tiered, multipart method for tracking tumor heterogeneity across samples obtained from a subject at different timepoints, are, in some embodiments, performed on one or more computers. In particular embodiments, the steps of performing a screen (e.g., screen 125 shown in FIG. 1A), performing an intra-individual analysis (e.g., intra-individual analysis 128A or intra-individual analysis 128B shown in FIG. 1A), and performing a second analysis (e.g., second analysis 130 shown in FIG. 1A) are performed on one or more computers. The steps of performing an assay (e.g., assay 120A and/or assay 120B shown in FIG. 1A) are not performed on one or more computers.

In various embodiments, the performance of the screen, the intra-individual analysis, and/or the second analysis can be implemented in hardware or software, or a combination of both. In one embodiment, a machine-readable storage medium is provided, the medium comprising a data storage material encoded with machine readable data which, when using a machine programmed with instructions for using said data, is capable of displaying data (e.g., methylation data) and results of the screen, intra-individual analysis, and/or second analysis (e.g., tracked tumor heterogeneity). Such data can be used for a variety of purposes, such as determining an efficacy of a tumor therapeutic, or selecting a new intervention for the subject. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods disclosed herein, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

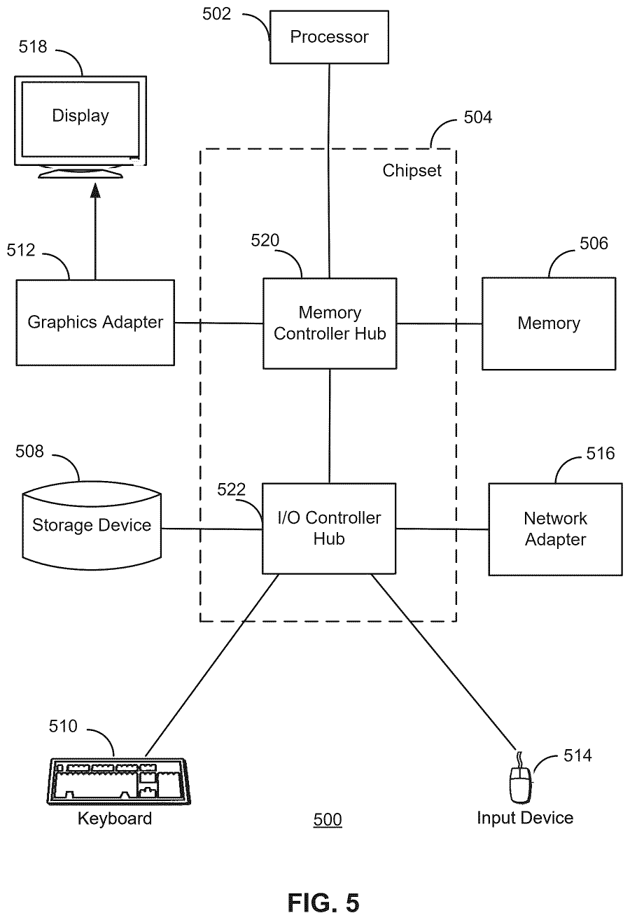

FIG. 5 illustrates an example computer for implementing the entities shown in FIGS. 1A-1C, 2A, 3A-3G, and 4A-4B. In particular embodiments, the example computer 500 can represent computational system 202 described in FIG. 2A. The computer 500 includes at least one processor 502 coupled to a chipset 504. The chipset 504 includes a memory controller hub 520 and an input/output (I/O) controller hub 422. A memory 506 and a graphics adapter 512 are coupled to the memory controller hub 520, and a display 518 is coupled to the graphics adapter 512. A storage device 508, an input device 514, and network adapter 516 are coupled to the I/O controller hub 522. Other embodiments of the computer 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input device 514 is a touch-screen interface, a mouse, track ball, or some combination thereof, and is used to input data into the computer 500. The keyboard 510 may be another device for inputting data into the computer 500. In some embodiments, the computer 500 may be configured to receive input (e.g., commands) from the input device 514 via gestures from the user. The graphics adapter 512 displays images and other information on the display 518. The network adapter 516 couples the computer 500 to one or more computer networks.

The computer 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 500 used by the entities of FIG. 1C can vary depending upon the embodiment and the processing power required by the entity. For example, the tumor heterogeneity system 170 can run in a single computer 500 or multiple computers 500 communicating with each other through a network such as in a server farm. The computers 500 can lack some of the components described above, such as graphics adapters 512, and displays 518.

### Kit Implementation

Also disclosed herein are kits for performing a tiered, multipart method for tracking tumor heterogeneity across samples obtained from a subject at different timepoints. Such kits can include equipment to draw a sample from a patient. For example, kits can include syringes and/or needles for obtaining a sample from a patient. Kits can include detection reagents for determining marker information using the sample obtained from the patient.

For example, detection reagents can include antibody reagents for performing a protein immunoassay. As another example, detection reagents can be a set of primers that, when combined with the sample, allows detection of a plurality of sites in cell-free DNA in the sample. In particular embodiments, the detection reagents enable detection of methylated or unmethylated target sites (e.g., methylated or unmethylated informative CpGs including one or more CGIs selected from Tables 1-4, or one or more CpGs within at least a portion of a region in Tables 1-4). Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. For example, the detection reagents may be primers that target specific known sequences of target sites, thereby enabling nucleic acid amplification of the target sites. Thus, the use of the detection reagents results in generation of methylation information of the patient corresponding to the target sites.

A kit can include instructions for use of one or more sets of detection reagents. For example, a kit can include instructions for performing at least one detection assay such as a nucleic acid amplification assay (e.g., polymerase chain reaction assay including any of real-time PCR assays, quantitative real-time PCR (qPCR) assays, allele-specific PCR assays, and reverse-transcription PCR assays), nucleic acid sequencing (e.g., targeted gene sequencing, targeted amplicon sequencing, whole genome sequencing, or whole genome bisulfite sequencing), hybrid capture, an immunoassay, a protein-binding assay, an antibody-based assay, an antigen-binding protein-based assay, a protein-based array, an enzyme-linked immunosorbent assay (ELISA), reporter assays, flow cytometry, a protein array, a blot, a Western blot, nephelometry, turbidimetry, chromatography, NMR, mass spectrometry, LC-MS, UPLC-MS/MS, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

Kits can further include instructions for accessing computer program instructions stored on a computer storage medium. In various embodiments, the computer program instructions, when executed by a processor of a computer system, cause the processor to perform one or more intra-individual analyses, generate background corrected methylation information, and/or track tumor heterogeneity across two or more timepoints.

In various embodiments, the kits include instructions for practicing the methods disclosed herein (e.g., performing an assay, screen, or diagnostic assay). These instructions can be present in the kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### Systems

Further disclosed herein are systems for performing a tiered, multipart method for tracking tumor heterogeneity across samples obtained from a subject at different timepoints. In various embodiments, such a system can include one or more sets of detection reagents for determining genomic information using a sample obtained from the patient, an apparatus configured to receive a mixture of the one or more sets of detection reagents and the sample obtained from a subject to generate methylation information of the subject, and a computer system communicatively coupled to the apparatus to generate background-corrected methylation information and/or to track the change in tumor heterogeneity.

The one or more sets of detection reagents enable the determination of marker information using the sample obtained from the patient. For example, detection reagents can include antibody reagents for performing a protein immunoassay. For example, detection reagents can be a set of primers that, when combined with the sample, allows detection of a plurality of sites in cell-free DNA in the sample. In particular embodiments, the detection reagents enable detection of methylated or methylated target sites (e.g., methylated or unmethylated informative CpGs including one or more CGI's selected from Tables 1-4 or one or more CpGs within at least a portion of a region in Tables 1-4). Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety.

The apparatus is configured to determine the methylation information from a mixture of the detection reagents and sample. For example, the apparatus can be configured to perform one or more of a nucleic acid amplification assay (e.g., polymerase chain reaction assay), nucleic acid sequencing (e.g., targeted gene sequencing, whole genome sequencing, or whole genome bisulfite sequencing), and hybrid capture to determine methylation information.

The mixture of the detection reagents and sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading. Examples of an apparatus include one or more of a sequencer, an incubator, plate reader (e.g., a luminescent plate reader, absorbance plate reader, fluorescence plate reader), a spectrometer, or a spectrophotometer.

The computer system, such as example computer 500 described in FIG. 5, communicates with the apparatus to receive the methylation information. The computer system generates background-corrected methylation information and can further track the change in tumor heterogeneity (e.g., based on the change of the background-corrected methylation information across two or more timepoints).

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., percentages, etc.), but some experimental error and deviation should be allowed for.

### Example 1: Overall Performance of Two-Tier Screening and Diagnosis of Patients with Prostate Cancer

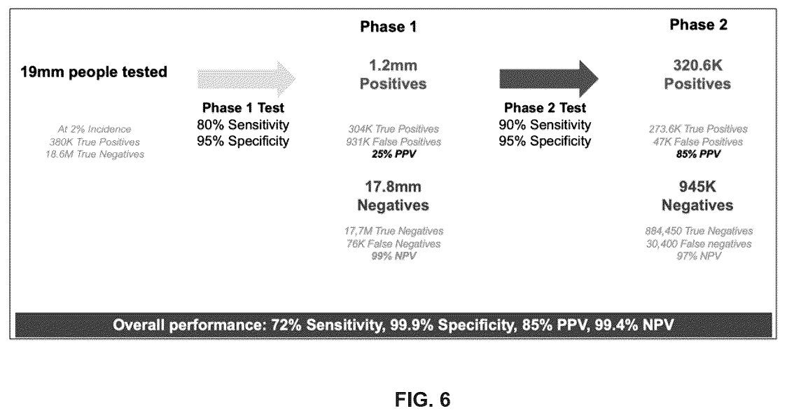

FIG. 6 shows example performance of different tiers of the multiple tier analysis for diagnosing individuals with cancer (e.g., prostate cancer). Here, the process begins with 19 million individuals who underwent testing. At a 2% incidence rate, of the 19 million individuals, 380,000 are true positives, and 18.6 million are true negatives.

The multi-tiered analysis involves performing a screen by analyzing methylation data (generated via an assay) of the patients. Here, the screen is designed to achieve 80% sensitivity and 95% specificity, thereby identifying 1.2 million out of the original 19 million individuals as at risk for prostate cancer. Additionally, the screen identifies 17.8 million out of the original 19 million individuals as not at risk for prostate cancer. Thus, these 17.8 million individuals need not undergo further analysis. Altogether, the screen achieves a 25% positive predictive rate and a 99% negative predictive rate.

The 1.2 million individuals identifies as at risk for prostate cancer further undergo a second test in the form of the second analysis. The second analysis achieves a 90% sensitivity and a 95% specificity. Of the 1.2 million individuals, −320,000 individuals are identified as having prostate cancer. This represents a 85% positive predictive rate as 273,600 individuals were true positives and 47,000 were false positives. Additionally, the second analysis identifies 945,000 negatives, of which 884,450 were true negatives, and 30,400 were false negatives, thereby representing a 97% negative predictive value.

Altogether, the overall performance of the multi-tier screen and second analysis includes 72% sensitivity, 99.9% specificity, 85% positive predictive value, and 99.4% negative predictive value.

Example steps for performing the multiple-tier analysis shown in FIG. 6 are detailed below.

### Prepare Target Specimen

The target specimen type (e.g. DNA, RNA, protein, exosomes, metabolites, etc.) is isolated from a patient's biological source (e.g. tissue, blood, plasma, serum, saliva, feces, etc.). That specimen can be isolated by a CRO or private or service laboratory or hospital or isolated internally using an internal procedure. Target specimens are assayed for quality and quantity measurements.

### Phase 1 Testing

Phase 1 testing is a relatively quick, non-invasive assay with simple technology, using small amounts of the target specimen. The result of this assay can be both qualitative and quantitative. Phase 1 testing is typically lower specificity (e.g. 95% specificity, 5% false positives) but higher sensitivity (e.g. 80% sensitivity, 20% false negatives) in order to screen a large proportion of the testing population rapidly and inexpensively. The phase 1 assay will overall increase the incidence of the target population (e.g. diseased) for the phase 2 assay, which will then increase the positive predictive value (PPV). Examples of the Phase 1 assay include but are not limited to ELISA assays, PCR assays, Real-time PCR assays, Quantitative real-time PCR (qPCR) assays, Allele-specific PCR assays, Reverse-transcription PCR assays and reporter assays.

### Phase 2 Testing

Phase 2 testing is a more complex, potentially invasive assay with complex technology, potentially using larger amounts of the target specimen. The result of this assay is both qualitative and quantitative. Phase 2 testing is typically higher specificity (e.g. 95% specificity, 10% false positives) but lower sensitivity (e.g. 90% sensitivity, 10% false negatives) in order to limit false positives. By screening out a large volume of the testing population, the target population has higher target incidence than the general population, which increases positive predictive value (PPV).

### Phase 2 Protocol

Examples of the phase 2 assay include but are not limited to Next Generation Sequencing assays utilizing target enrichment technologies, targeted amplicon sequencing technologies, whole genome sequencing, and whole genome bisulfite sequencing.

The target specimen for library construction is dsDNA isolated from formalin-fixed paraffin-embedded (FFPE) tissue. Alternatively, cfDNA is isolated from blood. For FFPE, the dsDNA is first mechanically sheared by the Covaris instrument utilizing adaptive focused acoustics to a target insert size of 200 base pairs. Post-shearing, a solid-phase reversible immobilization (SPRI) selection is done to remove smaller DNA fragments remaining in solution. For blood DNA, cfDNA is isolated. The fragmented DNA is then end-repaired and A-tailed (ERAT) to produce 5′-phosphorylated, 3′-dA-tailed dsDNA fragments. After ERAT, dsDNA unique dual index adapters with 3′-dTMP overhangs are then ligated to 3′-dA-tailed dsDNA fragments. Indices allow for sample multiplex for the downstream assay. Post-ligation, a solid-phase reversible immobilization (SPRI) selection is done to remove unwanted DNA fragments, excess adapters and molecules. PCR amplification is performed with a high-fidelity, low-bias polymerase at 10 cycles. Post-PCR, a SPRI selection is done to remove unwanted DNA fragments, excess primers, excess adapters and excess molecules. After library construction, the library quality and quantity are evaluated using the Agilent TapeStation and Qubit Fluorometer, respectively.

Libraries that pass quality control checks move forward to target enrichment through hybridization capture. Target enrichment by hybridization capture is defined as a positive selection strategy to enrich low abundance regions of interest from NGS libraries, allowing for more accurate sequencing analysis of these target regions. Indexed libraries are multi-plexed and hybridized to a custom, sequence specific, biotinylated probeset. The vast excess of probes drives their hybridization to complementary library fragments. The library fragment-biotinylated probe hybrid is pulled down by streptavidin beads, thereby capturing the target regions of interest. The streptavidin bead-bound library is sequentially washed with buffers to remove non-specifically associated library fragments. Following washes and recovery of captured libraries, samples are enriched for on target fragments and depleted for off-target fragments. Depletion of off-target fragments reduces overall library yield, requiring post-capture library amplification by PCR. The final amplified library is enriched for regions of interest. The hybrid captured library quality and quantity is evaluated using the Agilent TapeStation and Qubit Fluorometer, respectively. Additionally, the enrichment efficiency is evaluated using an iSeq Sequencing run and calculation of percent of reads within target enrichment panel. Measuring percent on-target is a good first approximation of target enrichment efficiency because the reads aligning to the target enrichment (bait) region indicate efficient hybridization and subsequent capture.

Target enriched libraries that pass quality control checks move forward to NovaSeq sequencing. Captured libraries with non-overlapping indices from library construction are pooled to multiplex for sequencing. Sequencing is completed on the NovaSeq 6000 instrument using paired end 150×150 base sequencing with a 10% PhiX spike-in. Sequencing data generated is then demultiplexed utilizing the assigned index, aligned to the human genome and trimmed to enrich for insert sample data only. This cleaned-up data is then processed through a quality pipeline to collapse duplicate reads and evaluate the sequencing data generated. Once the data is collapsed, the data is processed through a proprietary biomarker analysis pipeline to identify differences from the reference alignment (e.g. mutations, chemical modifications, etc). A report is then generated with the specific biomarker analysis per sample that confirms the results of the phase 1 assay or identifies true false positives from the phase 1 assay.

### Phase 1 Protocol:

An example protocol of an Allele-specific Real-Time PCR assay is as follows:
1. This assay runs DNA samples in triplicate with 2 ng input in 5 uL for the reference and mutation assays.
2. Combine 900 nmol/L unspecific primer(s), 100 nmol/L target probe(s), 2× polymerase enzyme(s), 2×dNTPs, 2× passive reference dyes, 10 uL water and 2 ng sample DNA at a pre-specified reaction volume as the reference control assay.
3. Combine 450 nmol/L allele-specific primer(s), 100 nmol/L target probe(s), 2× polymerase enzyme(s), 2×dNTPs, 2× passive reference dyes, 10 uL water and 2 ng sample DNA at a pre-specified reaction volume as the mutation assay.
4. Mix each reaction 10× and centrifuge to collect volume at the bottom of the well or tube.
5. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
6. Cycle threshold (Ct) values are recorded by the system and exported into an analysis program (e.g. Excel).
7. Average the Ct values between sample replicates for the reference and mutation assays.
8. Calculate the ΔCt between the sample average allele-specific Ct minus the sample average unspecific (reference) Ct.
9. Positive mutation results are identified by the ΔCt cut off >3 cycles and will move forward to phase 2 testing.

Allele-specific real-time PCR can be performed by combining library DNA with PCR reagents and primers specific for target sequences. The primers are designed to have single-base discrimination between tumor and non-tumor sequences. Perform real-time PCR (or digital PCR) for 30-50 cycles and monitor the output for signal via fluorescence from amplified target DNA or probe sequence. Cycle threshold values (Ct) are recorded and exported for analysis. The delta-Ct between negative control, positive control, and sample are calculated to determine presence or absence of target tumor sequences. Slight modifications of this protocol will allow for end-point PCR detection of RNA or DNA of tumor sequences. Phase 1 detection will be designed to remove 90-95% of non-cancer patient samples from moving forward for further testing.

ELISA assay detection of target molecules can be performed by coating an immunoassay well with monoclonal antibody designed to specifically detect target molecules, followed by blocking against non-specific binding. Next, target sample is introduced to the well, incubated and washed away. Any bound target can then be bound by a polyclonal antibody specific for the target. Additional secondary antibodies with color or fluorescent tags can be used to detect the presence of target molecules.

### Interpreting Results for Phase 1 and Phase 2 Assays

Two positive signals from the phase 1 assay and phase 2 assay can be determined as a true positive sample with an 85% probability of being accurate.

One negative signal from the phase 1 assay can be determined as a true negative sample with a 99% probability of being accurate.

One positive signal from the phase 1 assay and one negative signal from the phase 2 assay can be determined as an indeterminate sample with a 97% probability of a false positive in phase 1 assay.

### Example 2: Two-Tier Analysis Achieves Improved Performance in Comparison to Single Tier Analysis

Samples were obtained from patients of a patient population with an assumed 1.3% cancer prevalence. In total, 1046 samples obtained from the patients underwent either a single tier analysis or a two-tier analysis. The performance metrics (as measured by specificity, positive predictive value (PPV), and negative predictive value (NPV)) of each of the methodologies were determined.

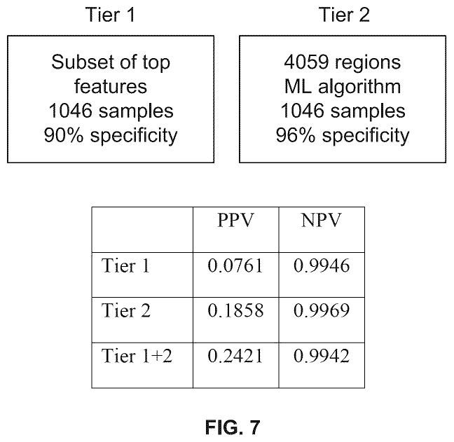

Reference is now made to FIG. 7, which depicts performance of a single tier and two-tier analysis of a population involving 1046 samples. The Tier 1 analysis focused on analyzing signal from a subset of the 4059 CGIs shown in Tables 2 and 3. In particular, 130 regions were analyzed to estimate tumor content according to methylation statuses of the regions, and estimated tumor content was used to distinguish patients that were negative or not negative for cancer. Logistic regression was performed to assess performance at 90% specificity (e.g., true negative rate reported as a proportion of correctly identified negatives). Performance was estimated to be about 63% sensitivity. For the single tier analysis (including only the Tier 1 analysis), it achieved a PPV (defined as number of true positives divided by the sum of true positives and false positives) of 0.0761 and a NPV (defined as true negative rate divided by the sum of true negatives and false negatives) of 0.9946. Thus, the single tier analysis was capable of successfully screening out a large proportion of samples that were negative for cancer. However, based on the low PPV, it had room for improvement in identifying samples that were true positives. The single tier analysis (including only a Tier 2 analysis) was additionally performed. Specifically, for each sample, signal of the 4059 CGIs was analyzed using a machine learning algorithm to distinguish samples having a cancer signal from samples not having a cancer signal. The single tier (Tier 2 analysis) achieved a PPV of 0.1858 and a NPV of 0.9969. Thus, the more costly Tier 2 analysis achieved a higher PPV in comparison to the less costly Tier 1 analysis without sacrificing the NPV metric.

Referring to the two-tier analysis, it involved performing the Tier 1 analysis (analyzing subset of top features) and samples deemed to be negative for cancer were screened out. An additional Tier 2 analysis was then performed. Specifically, for each sample, signal of the 4059 CGIs were analyzed using a machine learning algorithm to distinguish samples having a cancer signal from samples not having a cancer signal. Here, the Tier 2 analysis achieved a high specificity of 96%. For the two-tier analysis (including both the Tier 1 and Tier 2 analyses), the methodology achieved a PPV (defined as number of true positives divided by the sum of true positives and false positives) of 0.2421 and a NPV (defined as true negative rate divided by the sum of true negatives and false negatives) of 0.9942. Here, the two-tier analysis exhibited a significant improvement in comparison to the single-tier analysis. Specifically, the two-tier analysis achieved a higher specificity (e.g., 96% versus 90%). Furthermore, the two-tier analysis exhibited an improved PPV (0.2421 versus 0.0761) without adversely impacting the NPV (0.9942 versus 0.9946).

### Example 3: Example Samples and Assays for Conducting an Intra-Individual Analysis

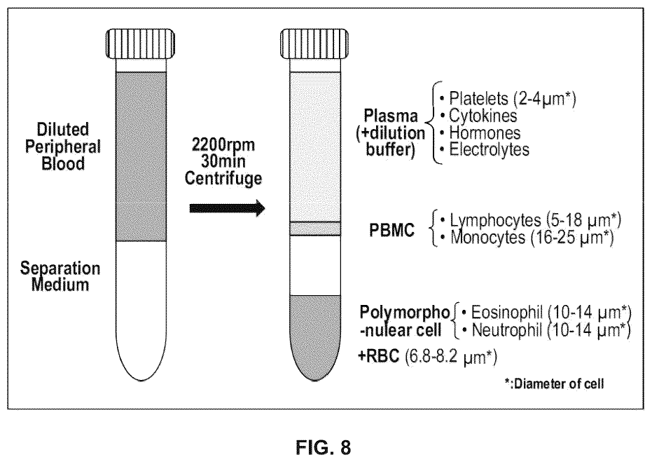

Blood samples are obtained from individuals. FIG. 8 shows an example sample from which target nucleic acids and reference nucleic acids are obtained. Shown on the left in FIG. 8 is a tube of blood obtained from an individual, the tube including diluted peripheral blood of the individual and separation medium. The tube undergoes centrifugation to separate different components of the diluted peripheral blood. For example, at a speed of 2200 rpm, the diluted peripheral blood is fractionated into plasma (including platelets, cytokines, hormones, and electrolytes), peripheral blood mononuclear cells (PBMCs), the separation medium, and polymorphonuclear cells. Here, target nucleic acids in the form of cell free DNA is found in the plasma whereas reference nucleic acids in the form of cellular genomic DNA is found in PBMCs.

Examples of an assay for generating sequence information from the target nucleic acids and the reference nucleic acids include but are not limited to Allele-specific PCR assays, Next Generation Sequencing assays, such as target enrichment technologies, targeted amplicon sequencing technologies, and whole genome sequencing.

An example protocol of an Allele-specific Real-Time PCR assay is as follows:
1. This assay runs all cfDNA samples in triplicate with 2 ng input in 5 uL for the reference and hypermethylation assays.
2. Combine 900 nmol/L unspecific primer(s), 100 nmol/L target probe(s), 2× polymerase enzyme(s), 2×dNTPs, 2× passive reference dyes, 10 uL water and 2 ng sample DNA at a pre-specified reaction volume as the reference control assay.
3. Combine 450 nmol/L allele-specific primer(s), 100 nmol/L target probe(s), 2× polymerase enzyme(s), 2×dNTPs, 2× passive reference dyes, 10 uL water and 2 ng sample DNA at a pre-specified reaction volume as the mutation assay.
4. Mix each reaction 10× and centrifuge to collect volume at the bottom of the well or tube.
5. Run the real-time PCR on a calibrated Real-Time PCR system under the following conditions: (1) 95° C. for 10 minutes followed by (2) 50 cycles of 90° C. for 15 seconds and
60° C. for 1 minute with fluorescence detection using FAM/VIC fluorophores.
6. Cycle threshold (Ct) values are recorded by the system and exported into an analysis program (e.g. Excel).
7. Average the Ct values between sample replicates for the reference and mutation assays.
8. Calculate the DCt between the sample average allele-specific Ct minus the sample average unspecific (reference) Ct.
9. Positive hypermethylation results are identified by the DCt cut off >3 cycles and will be compared to the patients individual PBMC natural signal.

An example protocol of an Allele-specific Real-Time PCR assay is as follows: Allele-specific real-time PCR can be performed by combining library from cfDNA with PCR reagents and primers specific for target sequences. The primers are designed to have single-base discrimination between tumor and non-tumor sequences. Perform real-time PCR (or digital PCR) for 30-50 cycles and monitor the output for signal via fluorescence from amplified target DNA or probe sequence. Cycle threshold values (Ct) are recorded and exported for analysis. The delta-Ct between negative control, positive control, and sample are calculated to determine presence or absence or absence of target tumor sequences. Slight modifications of this protocol will allow for end-point PCR detection of RNA or DNA of tumor sequences.

An example protocol of a next generation sequencing (NGS) Target Enrichment assay is as follows: The target specimen for library construction is dsDNA isolated from PBMCs. The dsDNA is first mechanically sheared by the Covaris instrument utilizing adaptive focused acoustics to a target insert size of 200 base pairs. Post-shearing, a solid-phase reversible immobilization (SPRI) selection is done to remove smaller DNA fragments remaining in solution. The fragmented DNA is then end-repaired and A-tailed (ERAT) to produce 5′-phosphorylated, 3′-dA-tailed dsDNA fragments. After ERAT, dsDNA unique dual index adapters with 3′-dTMP overhangs are then ligated to 3′-dA-tailed dsDNA fragments. Indices allow for sample multiplex for the downstream assay. Post-ligation, a solid-phase reversible immobilization (SPRI) selection is done to remove unwanted DNA fragments, excess adapters and molecules. PCR amplification is performed with a high-fidelity, low-bias polymerase at 10 cycles. Post-PCR, a SPRI selection is done to remove unwanted DNA fragments, excess primers, excess adapters and excess molecules. After library construction, the library quality and quantity are evaluated using the Agilent TapeStation and Qubit Fluorometer, respectively.

Libraries that pass quality control checks move forward to target enrichment through hybridization capture. Target enrichment by hybridization capture is defined as a positive selection strategy to enrich low abundance regions of interest from NGS libraries, allowing for more accurate sequencing analysis of these target regions. Indexed libraries are multi-plexed and hybridized to a custom, sequence specific, biotinylated probeset. The vast excess of probes drives their hybridization to complementary library fragments. The library fragment-biotinylated probe hybrid is pulled down by streptavidin beads, thereby capturing the target regions of interest. The streptavidin bead-bound library is sequentially washed with buffers to remove non-specifically associated library fragments. Following washes and recovery of captured libraries, samples are enriched for on target fragments and depleted for off-target fragments. Depletion of off-target fragments reduces overall library yield, requiring post-capture library amplification by PCR. The final amplified library is enriched for regions of interest. The hybrid captured library quality and quantity is evaluated using the Agilent TapeStation and Qubit Fluorometer, respectively. Additionally, the enrichment efficiency is evaluated using an iSeq Sequencing run and calculation of percent of reads within target enrichment panel. Measuring percent on-target is a good first approximation of target enrichment efficiency because the reads aligning to the target enrichment (bait) region indicate efficient hybridization and subsequent capture.

Target enriched libraries that pass quality control checks move forward to NovaSeq sequencing. Captured libraries with non-overlapping indices from library construction are pooled to multiplex for sequencing. Sequencing is completed on the NovaSeq 6000 instrument using paired end 150×150 base sequencing with a 10% PhiX spike-in. Sequencing data generated is then demultiplexed utilizing the assigned index, aligned to the human genome and trimmed to enrich for insert sample data only. This cleaned-up data is then processed through a quality pipeline to collapse duplicate reads and evaluate the sequencing data generated. Once the data is collapsed, the data is processed through a proprietary analysis pipeline to identify differences from the reference alignment (e.g. mutations, chemical modifications, etc.). A report is then generated with the specific signal informative for determining presence or absence of cancer.

1. A tiered, multipart method for tracking tumor heterogeneity across at least first and second biological samples obtained from a subject, the method comprising:
(a) performing a first analysis of nucleic acid sequence information that was derived from an assay performed on a first biological sample obtained from the subject at a first timepoint to identify whether the biological sample is not at risk of containing circulating tumor DNA;
(b) responsive to determining that the first biological sample is not identified as not at risk:
(i) performing a first intra-individual analysis using the first biological sample to generate a first set of background-corrected methylation information representing a difference between methylation information from target nucleic acids from the first biological sample and methylation information from reference nucleic acids from the first biological sample;
(ii) performing, a second intra-individual analysis using a second biological sample to generate a second set of background-corrected methylation information representing a difference between methylation information from target nucleic acids from the second biological sample and methylation information from reference nucleic acids from the second biological sample, wherein the second biological sample was obtained from the subject at a second timepoint subsequent to the first timepoint;
(iii) determining a change in signal between the first set of background-corrected methylation information from the first intra-individual analysis and the second set of background-corrected methylation information from the second intra-individual analysis; and
(iv) performing a second analysis comprising analyzing the determined change in signal to track tumor heterogeneity across the first biological sample and the second biological sample.

2. A method for tracking tumor heterogeneity in a patient during or subsequent to administration of a tumor therapeutic, or in a patient being considered for administration of a tumor therapeutic, comprising:
(a) confirming that a first biological sample of the patient is not identified as not at risk of containing circulating tumor DNA;
(b) responsive to determining that the first biological sample is not identified as not at risk:
(i) performing, at a baseline timepoint, a first intra-individual analysis using the first biological sample to generate a first set of background-corrected methylation information representing a difference between methylation information from target nucleic acids from the first biological sample and methylation information from reference nucleic acids from the first biological sample;
(ii) performing, at a second timepoint, a second intra-individual analysis using a second biological sample to generate a second set of background-corrected methylation information representing a difference between methylation information from target nucleic acids from the second biological sample and methylation information from reference nucleic acids from the second biological sample, wherein between the baseline timepoint and second timepoint the patient may be administered, or continues to be administered, one or more tumor therapeutics;
(iii) determining a change in signal between the first set of background-corrected methylation information from the first intra-individual analysis and the second set of background-corrected methylation information from the second intra-individual analysis; and
(iv) performing a second analysis comprising analyzing the determined change in signal to assess tumor heterogeneity across the first biological sample and the second biological sample and therefore track the patient's therapeutic progress and/or assess the tumor therapeutic.

3. The method of claim 1 or 2, wherein determining the change in signal comprises determining a difference between the first set of background-corrected methylation information from the first intra-individual analysis and the second set of background-corrected methylation information from the second intra-individual analysis.

4. The method of any one of claims 1-3, wherein the first set of background-corrected methylation information or the second set of background-corrected methylation information comprises methylation statuses for a plurality of genomic sites.

5. The method of claim 4, wherein the plurality of genomic sites comprise a plurality of CpG sites.

6. The method of claim 5, wherein the plurality of CpG sites are located in one or more CpG islands or portions of one or more CpG islands shown in Tables 1-4.

7. The method of claim 4, wherein the first set of background-corrected methylation information and the second set of background-corrected methylation information comprises methylation statuses for a plurality of CpG sites.

8. The method of claim 7, wherein the plurality of CpG sites of the first set of background-corrected methylation information are the same plurality of CpG sites of the second set of background-corrected methylation information.

9. The method of any one of claims 1-8, wherein performing the first intra-individual analysis comprises:
obtaining target nucleic acids and reference nucleic acids from the first biological sample obtained from the subject;
performing bisulfite conversion of the target nucleic acids and the reference nucleic acids;
selectively amplifying target regions comprising a plurality of CpG sites of the bisulfite converted target nucleic acids and reference nucleic acids;
generating a dataset comprising methylation information of the plurality of CpG sites from the target nucleic acids and methylation information of the plurality of CpG sites from the reference nucleic acids; and
using a computer processor, combining the methylation information of the plurality of CpG sites from the target nucleic acids and the methylation information of the plurality of CpG sites from the reference nucleic acids to generate the first set of background-corrected methylation information.

10. The method of claim 9, wherein the reference nucleic acids from the first biological sample comprise genomic DNA from peripheral blood mononuclear cells (PBMCs) or polymorphonuclear cells of the subject.

11. The method of any one of claims 1-10, wherein the first set of background-corrected methylation information comprises phased sequencing information.

12. The method of claim 11, wherein the phased sequencing information of the first set of background-corrected methylation information is generated by:
obtaining or having obtained sequence reads of cell-free DNA from the first sample;
obtaining or having obtained long sequence reads of reference nucleic acids from the second sample, wherein the long sequence reads of reference nucleic acids are at least 500 bases in length;
attributing long sequence reads of reference nucleic acids to one of two or more different sources of the subject; and
aligning the obtained sequence reads of cell-free DNA to the long sequence reads of reference nucleic acids.

13. The method of claim 12, wherein the phased sequencing information of cell-free DNA comprises methylation statuses for a plurality of genomic sites of the cell-free DNA.

14. The method of claim 13, wherein the methylation statuses for the plurality of genomic sites comprise at least one coupled genomic site representing two or more methylated genomic sites originating from a common source.

15. The method of claim 12, wherein the phased sequencing information comprises mutation sequence information of the cell-free DNA.

16. The method of claim 15, wherein the mutation sequence information comprises a plurality of mutations present across the plurality of genomic sites.

17. The method of claim 16, wherein the plurality of mutations present across the plurality of genomic sites comprise coupled genomic sites representing two or more mutated genomic sites originating from a common source.

18. The method of claim 16 or 17, wherein the plurality of mutations comprise one or more of a single nucleotide polymorphism (SNP), single nucleotide variant (SNV), insertion, deletion, copy number variation (CNV), duplication, or translocation.

19. The method of any one of claims 12-18, wherein the two or more different sources of the subject comprise a maternal chromosome source or a paternal chromosome source.

20. The method of any one of claims 12-19, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases.

21. The method of any one of claims 1-8, wherein performing the second intra-individual analysis comprises:
obtaining target nucleic acids and reference nucleic acids from the second biological sample obtained from the subject;
performing bisulfite conversion of the target nucleic acids and the reference nucleic acids;
selectively amplifying target regions comprising a plurality of CpG sites of the bisulfite converted target nucleic acids and reference nucleic acids;
generating a dataset comprising methylation information of the plurality of CpG sites from the target nucleic acids and methylation information of the plurality of CpG sites from the reference nucleic acids; and
using a computer processor, combining the methylation information of the plurality of CpG sites from the target nucleic acids and the methylation information of the plurality of CpG sites from the reference nucleic acids to generate the second set of background-corrected methylation information.

22. The method of claim 21, wherein the reference nucleic acids from the second biological sample comprise genomic DNA from peripheral blood mononuclear cells (PBMCs) or polymorphonuclear cells of the subject.

23. The method of any one of claims 1-22, wherein the first set of background-corrected methylation information and/or the second set of background-corrected methylation information comprise a high resolution measure of methylation.

24. The method of claim 23, wherein the high resolution measure of methylation comprises a total quantity of consecutively methylated CpG sites within target regions.

25. The method of claim 24, wherein the total quantity of consecutively methylated CpG sites within target regions comprises the total quantity of 3, 4, or 5 consecutively methylated CpG sites within target regions.

26. The method of claim 23, wherein the high resolution measure of methylation comprises methylation statuses of a plurality of CpG sites from a haplotype.

27. The method of any one of claims 1-26, wherein the second set of background-corrected methylation information comprises phased sequencing information.

28. The method of claim 27, wherein the phased sequencing information of the second set of background-corrected methylation information is generated by:
obtaining or having obtained sequence reads of cell-free DNA from the second sample;
obtaining or having obtained long sequence reads of reference nucleic acids from the second sample, wherein the long sequence reads of reference nucleic acids are at least 500 bases in length;
attributing long sequence reads of reference nucleic acids to one of two or more different sources of the subject; and
aligning the obtained sequence reads of cell-free DNA to the long sequence reads of reference nucleic acids.

29. The method of claim 28, wherein the phased sequencing information of cell-free DNA comprises methylation statuses for a plurality of genomic sites of the cell-free DNA.

30. The method of claim 29, wherein the methylation statuses for the plurality of genomic sites comprise at least one coupled genomic site representing two or more methylated genomic sites originating from a common source.

31. The method of claim 28, wherein the phased sequencing information comprises mutation sequence information of the cell-free DNA.

32. The method of claim 31, wherein the mutation sequence information comprises a plurality of mutations present across the plurality of genomic sites.

33. The method of claim 32, wherein the plurality of mutations present across the plurality of genomic sites comprise coupled genomic sites representing two or more mutated genomic sites originating from a common source.

34. The method of claim 32 or 33, wherein the plurality of mutations comprise one or more of a single nucleotide polymorphism (SNP), single nucleotide variant (SNV), insertion, deletion, copy number variation (CNV), duplication, or translocation.

35. The method of any one of claims 28-34, wherein the two or more different sources of the subject comprise a maternal chromosome source or a paternal chromosome source.

36. The method of any one of claims 28-35, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases.

37. The method of any one of claims 1-36, wherein the nucleic acid sequence information of the first analysis comprises methylation sequence information.

38. The method of claim 37, wherein the methylation sequence information of the first analysis comprises methylation statuses for a plurality of genomic sites.

39. The method of claim 38, wherein the plurality of genomic sites comprise a plurality of CpG sites.

40. The method of claim 38, wherein the nucleic acid sequence information of the first analysis comprises a measure of overall methylation across the plurality of genomic sites.

41. The method of claim 40, wherein the measure of overall methylation comprises a total number of methylated genomic sites or an average number of methylated genomic sites.

42. The method of any one of claims 1-41, wherein performing the first analysis of nucleic acid sequence information comprises applying a trained machine learning model.

43. The method of any one of claims 1-42, wherein the method delivers improved performance as a function of resource consumption in comparison to the single tier method.

44. The method of any one of claims 1-42, wherein the method achieves an improved performance metric in comparison to a single tier method.

45. The method of any one of claims 1-42, wherein the method tracks tumor heterogeneity of one or more of the early stage cancers.

46. The method of claim 45, wherein the one or more of the early stage cancers is acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

47. The method of claim 45, wherein the one or more early stage cancers is a preclinical phase cancer.

48. The method of claim 47, wherein the preclinical phase cancer is stage I or stage II cancer.

49. The method of any one of claims 1-48, wherein the nucleic acid sequence information, the background-corrected methylation information of the first intra-individual analysis, and/or the background-corrected methylation information of the second intra-individual analysis is obtained from an assay, wherein the assay comprises performing one or more of:
a. sequencing of nucleic acids;
b. hybrid capture;
c. methylation-specific PCR;
d. an assay that generates methylation information; and
e. sequencing a clone library generated from a template immortalized library.

50. The method of any one of claims 1-49, wherein each of the first biological sample and the second biological sample independently comprises any one of a blood sample, a stool sample, a urine sample, a mucous sample, or a saliva sample.

51. The method of any one of claims 1-50, wherein each of the first biological sample and the second biological sample is a blood sample.

52. The method of claim 51, wherein each of the first biological sample and the second biological sample does not comprise an invasive biopsy sample.

53. The method of any one of claims 1-52, wherein the second analysis comprises whole genome sequencing, optionally whole genome bisulfite sequencing.

54. The method of any one of claim 1-53, wherein the subject received a tumor therapeutic prior to the first timepoint.

55. The method of any one of claim 1-53, wherein subsequent to the first timepoint and prior to the second timepoint, the subject received a tumor therapeutic.

56. The method of claim 54 or 55, further comprising determining an efficacy of the tumor therapeutic based on the tracked tumor heterogeneity.

57. The method of claim 56, wherein if the tracked tumor heterogeneity indicates a stable or increasing tumor heterogeneity in the subject across the first biological sample and the second biological sample, determining that the tumor therapeutic lacks efficacy.

58. The method of claim 57, further comprising selecting a new tumor therapeutic for the subject responsive to determining that the tumor therapeutic lacks efficacy.

59. The method of claim 56, wherein if the tracked tumor heterogeneity indicates a reducing tumor heterogeneity in the subject across the first biological sample and the second biological sample, determining that the tumor therapeutic achieves therapeutic efficacy.

60. The method of any one of claims 1-59, wherein prior to (a), a prior sample obtained from the subject was previously determined to be not at risk for containing circulating tumor DNA.

61. The method of claim 60, wherein further responsive to determining that the first biological sample is not identified as not at risk, determining that the prior sample previously determined to be not at risk for containing circulating tumor DNA was a false negative.

62. The method of claim 60, wherein if the tracked tumor heterogeneity indicates an increasing tumor heterogeneity in the subject across the first biological sample and the second biological sample, determining that the prior sample previously determined to be not at risk for containing circulating tumor DNA was a false negative.

63. A tiered, multipart method for assessing tumor heterogeneity across at least first and second biological samples obtained from a subject, the method comprising:
(a) performing a first analysis of nucleic acid sequence information that was derived from an assay performed on a first biological sample obtained at a first timepoint to identify whether the biological sample is not at risk of containing circulating tumor DNA,
(b) responsive to determining that the first biological sample is not identified as not at risk:
(i) performing a first intra-individual analysis using the first biological sample to generate a first set of background-corrected methylation information representing a difference between methylation information from target nucleic acids from the first biological sample and methylation information from reference nucleic acids from the first biological sample;
(ii) performing a second analysis of the first biological sample comprising analyzing the background-corrected methylation information to predict a tumor heterogeneity state;
(c) determining an updated tumor heterogeneity state by:
(i) performing a second intra-individual analysis using a second biological sample to generate a second set of background-corrected methylation information representing a difference between methylation information from target nucleic acids from the second biological sample and methylation information from reference nucleic acids from the second biological sample, wherein the second biological sample was obtained from the subject at a second timepoint subsequent to the first timepoint; and
(ii) performing a second analysis of the second biological sample comprising analyzing the background-corrected methylation information to predict the updated tumor heterogeneity state; and
(d) comparing the tumor heterogeneity state from the first biological sample to the updated tumor heterogeneity state from the second biological sample to track tumor heterogeneity across the first biological sample and the second biological sample.

64. A method for assessing tumor heterogeneity in a patient during or subsequent to administration of a tumor therapeutic, or in a patient being considered for administration of a tumor therapeutic, comprising:
(a) confirming that a first biological sample of the patient is not identified as not at risk of containing circulating tumor DNA;
(b) responsive to determining that the first biological sample is not identified as not at risk:
(i) performing, at a baseline timepoint, a first intra-individual analysis using the first biological sample to generate a first set of background-corrected methylation information representing a difference between methylation information from target nucleic acids from the first biological sample and methylation information from reference nucleic acids from the first biological sample;
(ii) performing, at a second timepoint, a second intra-individual analysis using a second biological sample to generate a second set of background-corrected methylation information representing a difference between methylation information from target nucleic acids from the second biological sample and methylation information from reference nucleic acids from the second biological sample, wherein between the baseline timepoint and second timepoint the patient may be administered, or continues to be administered, one or more tumor therapeutics;
(iii) determining a change in signal between the first set of background-corrected methylation information from the first intra-individual analysis and the second set of background-corrected methylation information from the second intra-individual analysis; and
(iv) performing a second analysis comprising analyzing the determined change in signal to assess tumor heterogeneity across the first biological sample and the second biological sample and therefore track the patient's therapeutic progress and/or assess the tumor therapeutic.

65. The method of claim 63 or 64, wherein the first set of background-corrected methylation information or the second set of background-corrected methylation information comprises methylation statuses for a plurality of genomic sites.

66. The method of claim 65, wherein the plurality of genomic sites comprise a plurality of CpG sites.

67. The method of claim 66, wherein the plurality of CpG sites are located in one or more CpG islands or portions of one or more CpG islands shown in Tables 1-4.

68. The method of claim 65, wherein the first set of background-corrected methylation information and the second set of background-corrected methylation information comprises methylation statuses for the plurality of CpG sites.

69. The method of claim 68, wherein the plurality of CpG sites of the first set of background-corrected methylation information are the same plurality of CpG sites of the second set of background-corrected methylation information.

70. The method of any one of claims 63-69, wherein performing the first intra-individual analysis comprises:
obtaining target nucleic acids and reference nucleic acids from the first biological sample obtained from the subject;
performing bisulfite conversion of the target nucleic acids and the reference nucleic acids;
selectively amplifying target regions comprising a plurality of CpG sites of the bisulfite converted target nucleic acids and reference nucleic acids;
generating a dataset comprising methylation information of the plurality of CpG sites from the target nucleic acids and methylation information of the plurality of CpG sites from the reference nucleic acids; and
using a computer processor, combining the methylation information of the plurality of CpG sites from the target nucleic acids and the methylation information of the plurality of CpG sites from the reference nucleic acids to generate the first set of background-corrected methylation information.

71. The method of claim 70, wherein the reference nucleic acids from the first biological sample comprise genomic DNA from peripheral blood mononuclear cells (PBMCs) or polymorphonuclear cells of the subject.

72. The method of any one of claims 63-71, wherein the first set of background-corrected methylation information comprise a high resolution measure of methylation.

73. The method of claim 72, wherein the high resolution measure of methylation comprises a total quantity of consecutively methylated CpG sites within target regions.

74. The method of claim 73, wherein the total quantity of consecutively methylated CpG sites within target regions comprises the total quantity of 3, 4, or 5 consecutively methylated CpG sites within target regions.

75. The method of claim 72, wherein the high resolution measure of methylation comprises methylation statuses of a plurality of CpG sites from a haplotype.

76. The method of any one of claims 63-75, wherein the first set of background-corrected methylation information comprises phased sequencing information.

77. The method of claim 76, wherein the phased sequencing information of the first set of background-corrected methylation information is generated by:
obtaining or having obtained sequence reads of cell-free DNA from the first sample;
obtaining or having obtained long sequence reads of reference nucleic acids from the second sample, wherein the long sequence reads of reference nucleic acids are at least 500 bases in length;
attributing long sequence reads of reference nucleic acids to one of two or more different sources of the subject; and
aligning the obtained sequence reads of cell-free DNA to the long sequence reads of reference nucleic acids.

78. The method of claim 77, wherein the phased sequencing information comprises methylation statuses for a plurality of genomic sites.

79. The method of claim 78, wherein the methylation statuses for the plurality of genomic sites comprise at least one coupled genomic site representing two or more methylated genomic sites originating from a common source.

80. The method of claim 77, wherein the phased sequencing information comprises mutation sequence information of the cell-free DNA.

81. The method of claim 80, wherein the mutation sequence information comprises a plurality of mutations present across the plurality of genomic sites.

82. The method of claim 81, wherein the plurality of mutations present across the plurality of genomic sites comprise coupled genomic sites representing two or more mutated genomic sites originating from a common source.

83. The method of claim 81 or 82, wherein the plurality of mutations comprise one or more of a single nucleotide polymorphism (SNP), single nucleotide variant (SNV), insertion, deletion, copy number variation (CNV), duplication, or translocation.

84. The method of any one of claims 77-83, wherein the two or more different sources of the subject comprise a maternal chromosome source or a paternal chromosome source.

85. The method of any one of claims 77-84, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases.

86. The method of any one of claims 63-69, wherein performing the second intra-individual analysis comprises:
obtaining target nucleic acids and reference nucleic acids from the second biological sample obtained from the subject;
performing bisulfite conversion of the target nucleic acids and the reference nucleic acids;
selectively amplifying target regions comprising a plurality of CpG sites of the bisulfite converted target nucleic acids and reference nucleic acids;
generating a dataset comprising methylation information of the plurality of CpG sites from the target nucleic acids and methylation information of the plurality of CpG sites from the reference nucleic acids; and
using a computer processor, combining the methylation information of the plurality of CpG sites from the target nucleic acids and the methylation information of the plurality of CpG sites from the reference nucleic acids to generate the first set of background-corrected methylation information.

87. The method of claim 86, wherein the reference nucleic acids from the second biological sample comprise genomic DNA from peripheral blood mononuclear cells (PBMCs) or polymorphonuclear cells of the subject.

88. The method of any one of claims 86-87, wherein the second set of background-corrected methylation information comprises a high resolution measure of methylation.

89. The method of claim 88, wherein the high resolution measure of methylation comprises a total quantity of consecutively methylated CpG sites within target regions.

90. The method of claim 89, wherein the total quantity of consecutively methylated CpG sites within target regions comprises the total quantity of 3, 4, or 5 consecutively methylated CpG sites within target regions.

91. The method of claim 88, wherein the high resolution measure of methylation comprises methylation statuses of a plurality of CpG sites from a haplotype.

92. The method of any one of claims 63-91, wherein the second set of background-corrected methylation information comprises phased sequencing information.

93. The method of claim 92, wherein the phased sequencing information of the second set of background-corrected methylation information is generated by:
obtaining or having obtained sequence reads of cell-free DNA from the second sample;
obtaining or having obtained long sequence reads of reference nucleic acids from the second sample, wherein the long sequence reads of reference nucleic acids are at least 500 bases in length;
attributing long sequence reads of reference nucleic acids to one of two or more different sources of the subject; and
aligning the obtained sequence reads of cell-free DNA to the long sequence reads of reference nucleic acids.

94. The method of claim 93, wherein the phased sequencing information of cell-free DNA comprises methylation statuses for a plurality of genomic sites of the cell-free DNA.

95. The method of claim 94, wherein the methylation statuses for the plurality of genomic sites comprise at least one coupled genomic site representing two or more methylated genomic sites originating from a common source.

96. The method of claim 93, wherein the phased sequencing information comprises mutation sequence information of the cell-free DNA.

97. The method of claim 96, wherein the mutation sequence information comprises a plurality of mutations present across the plurality of genomic sites.

98. The method of claim 97, wherein the plurality of mutations present across the plurality of genomic sites comprise coupled genomic sites representing two or more mutated genomic sites originating from a common source.

99. The method of claim 97 or 98, wherein the plurality of mutations comprise one or more of a single nucleotide polymorphism (SNP), single nucleotide variant (SNV), insertion, deletion, copy number variation (CNV), duplication, or translocation.

100. The method of any one of claims 93-99, wherein the two or more different sources of the subject comprise a maternal chromosome source or a paternal chromosome source.

101. The method of any one of claims 93-100, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases.

102. The method of any one of claims 63-101, wherein the nucleic acid sequence information of the first analysis comprises methylation sequence information.

103. The method of claim 102, wherein the methylation sequence information of the first analysis comprises methylation statuses for a plurality of genomic sites.

104. The method of claim 103, wherein the plurality of genomic sites comprise a plurality of CpG sites.

105. The method of claim 103, wherein the nucleic acid sequence information of the first analysis comprises a measure of overall methylation across the plurality of genomic sites.

106. The method of claim 105, wherein the measure of overall methylation comprises a total number of methylated genomic sites or an average number of methylated genomic sites.

107. The method of any one of claims 63-106, wherein performing the first analysis of nucleic acid sequence information comprises applying a trained machine learning model.

108. The method of any one of claims 63-107, wherein the tiered, multipart method delivers improved performance as a function of resource consumption in comparison to the single tier method.

109. The method of any one of claims 63-107, wherein the tiered, multipart method achieves an improved performance metric in comparison to a single tier method.

110. The method of any one of claims 63-107, wherein the tiered, multipart method tracks tumor heterogeneity of one or more of the early stage cancers.

111. The method of claim 110, wherein the one or more of the early stage cancers is acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

112. The method of claim 110, wherein the one or more early stage cancers is a preclinical phase cancer.

113. The method of claim 112, wherein the preclinical phase cancer is stage I or stage II cancer.

114. The method of any one of claims 63-113, wherein the nucleic acid sequence information, the background-corrected methylation information of the first intra-individual analysis, and/or the background-corrected methylation information of the second intra-individual analysis is obtained from an assay, wherein the assay comprises performing one or more of:
a. sequencing of nucleic acids;
b. hybrid capture;
c. methylation-specific PCR;
d. an assay that generates methylation information; and
e. sequencing a clone library generated from a template immortalized library.

115. The method of any one of claims 63-114, wherein each of the first biological sample and the second biological sample independently comprises any one of a blood sample, a stool sample, a urine sample, a mucous sample, or a saliva sample.

116. The method of any one of claims 63-115, wherein each of the first biological sample and the second biological sample is a blood sample.

117. The method of claim 116, wherein each of the first biological sample and the second biological sample does not comprise an invasive biopsy sample.

118. The method of any one of claims 63-117, wherein the second analysis of the first biological sample or the second analysis of the second biological sample comprises whole genome sequencing, optionally whole genome bisulfite sequencing.

119. The method of any one of claim 63-118, wherein the subject received a tumor therapeutic prior to the first timepoint.

120. The method of any one of claim 63-118, wherein subsequent to the first timepoint and prior to the second timepoint, the subject received a tumor therapeutic.

121. The method of claim 119 or 120, further comprising determining an efficacy of the tumor therapeutic based on the assessed tumor heterogeneity.

122. The method of claim 121, wherein if the tracked tumor heterogeneity indicates a stable or increasing tumor heterogeneity in the subject across the first biological sample and the second biological sample, determining that the assessed tumor therapeutic lacks efficacy.

123. The method of claim 122, further comprising selecting a new intervention for the subject responsive to determining that the assessed tumor therapeutic lacks efficacy.

124. The method of claim 121, wherein if the tracked tumor heterogeneity indicates a reducing tumor heterogeneity in the subject across the first biological sample and the second biological sample, determining that the assessed tumor therapeutic achieves therapeutic efficacy.

125. The method of any one of claims 63-124, wherein prior to (a), a prior sample obtained from the subject was previously determined to be not at risk for containing circulating tumor DNA.

126. The method of claim 125, wherein further responsive to determining that the first biological sample is not identified as not at risk, determining that the prior sample previously determined to be not at risk for containing circulating tumor DNA was a false negative.

127. The method of claim 125, wherein if the tracked tumor heterogeneity indicates an increasing tumor heterogeneity in the subject across the first biological sample and the second biological sample, determining that the prior sample previously determined to be not at risk for containing circulating tumor DNA was a false negative.

128. A tiered, multipart method for determining whether a prior sample obtained from a subject was a false negative sample, the method comprising:
(a) performing a first analysis of nucleic acid sequence information that was derived from an assay performed on a first biological sample obtained from the subject at a first timepoint to identify whether the biological sample is not at risk of containing circulating tumor DNA, wherein the prior sample was obtained from the subject prior to the first biological sample,
(b) responsive to determining that the first biological sample is not identified as not at risk:
(i) performing one or more intra-individual analyses using the first biological sample or one or more additional biological samples, wherein performing each intra-individual analysis involves generating background-corrected methylation information representing a difference between methylation information from target nucleic acids and methylation information from reference nucleic acids from one of the first biological sample or one or more additional biological samples;
(ii) performing a longitudinal analysis comprising analyzing generated background-corrected methylation information from each of the one or more intra-individual analyses; and
(iii) determining that the prior sample obtained from a subject was a false negative sample.

129. The method of claim 128, wherein determining that the prior sample obtained from a subject was a false negative sample is responsive to determining that the first biological sample is not identified as not at risk.

130. The method of claim 128, wherein determining that the prior sample obtained from a subject was a false negative sample is responsive to performing the longitudinal analysis.

131. The method of any one of claims 128-130, wherein performing the longitudinal analysis comprising tracking tumor heterogeneity across the first biological sample and one or more additional biological samples.

132. A non-transitory computer readable medium comprising instructions that, when executed by a processor, cause the processor to perform the method of any one of claims 1-127.

133. A system comprising:
a processor; and
a non-transitory computer readable medium comprising instructions that, when executed by a processor, cause the processor to perform the method of any one of claims 1-127.
